(19) United States
(12) Patent Application Publication
(10) Pub. No.: US 2009/0176847 A1
(43) Pub. Date: Jul. 9, 2009

(54) Dicyclooctane Derivatives, Preparation Processes and Medical Uses Thereof

(76) Inventors: Peng Cho Tang, Minhang District (CN); Zhigang Lin, Minhang District (CN); Lei Zhang, Minhang District (CN); Qian Wang, Minhang District (CN); Jialiang Yang, Minhang District (CN); Yang Wang, Minhang District (CN); Fuqiang Zhao, Minhang District (CN)

Correspondence Address: BINGHAM MCCUTCHEN LLP, 2020 K Street, N.W., Intellectual Property Department, WASHINGTON, DC, 20006, US

(21) Appl. No.: 12/226,011

(22) PCT Filed: Mar. 28, 2007
(86) PCT No.: PCT/CN2007/001008

§ 371 Date: Oct. 3, 2008

(30) Foreign Application Priority Data
Apr. 5, 2006 (CN) 200610066400.8

(51) Int. Cl. A61K 31/426; C07D 207/16; A61K 31/40; C07D 277/06; A61P 3/10; C07C 49/345

(52) U.S. Cl. 514/365; 548/540; 514/423; 548/200; 568/303

(57) ABSTRACT

The present invention relates to new dicyclooctane derivates represented by general formula (I), preparation processes and pharmaceutical compositions containing them, and to uses for treatment especially for dipeptidyl peptidase inhibitor (DPPIV), in which each substituent group of general formula (I) is as defined in specification.

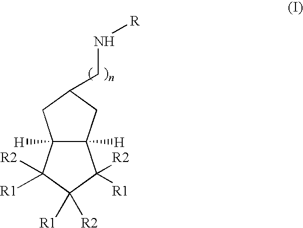

### FIELD OF THE INVENTION

This invention relates to novel bicyclooctane derivatives, methods for their preparation, pharmaceutical compositions containing them and therapeutic use thereof, particularly their pharmaceutical use as dipeptidyl peptidase IV inhibitor.

### BACKGROUND OF THE INVENTION

Diabetes refers to a disease process derived from multiple causative factors and characterized by elevated levels of plasma glucose or hyperglycemia along with sugar, fat and protein metabolism disorder caused by insulin secretion and/or the action defects. Diabetes is an ancient disease, and due to the human body absolute or relative lack of insulin resulting in increased concentrations of glucose in the blood which largely discharges in urine with more drink, more urine, more food, weight loss, dizziness, weakness and other symptoms.

Dipeptidyl peptidase-IV (DPPIV) is a serine protease which cleaves N-terminal dipeptides from a peptide chain containing, preferably, a proline residue in the penultimate position. Although the biological role of DPPIV in mammalian systems has not been completely established, it is believed to play an important role in neuropeptide metabolism, T-cell activation, attachment of cancer cells to the endothelium and the entry of HIV into lymphoid cells (WO98/19998).

More recently, it was discovered that DPPIV is responsible for inhibiting the secretion of glucagon-like peptide (GLP)-1. More particularly, DPPIV cleaves the amino-terminal His-Ala dipeptide of GLP-1, degrading active GLP-1(7-36)NH₂ into inactive GLP-1(9-36)NH₂ (Endocrinology, 1999, 140: 5356-5363). Under the physiological condition, the half-life of GLP-1 is short, the inactive metabolite from GPP-1 degraded by DPPIV can combine with GLP-1 receptor to antagonize the active GLP-1, so the physiological response to GPL-1 is reduced. The endogenous even exogenous GLP-1 can be entirely protected by the DPPIV inhibitor from being deactivated by DPPIV, and the GLP-1 bioactivity can be significantly increased (5- to 10-fold). Since GLP-1 is a major stimulator of pancreatic insulin secretion and can directly effect on glucose disposal, the DPPIV inhibitor is well useful for treating non-insulin-dependent diabetes mellitus (NIDDM) (U.S. Pat. No. 6,110,949).

### SUMMARY OF THE INVENTION

Accordingly, the present invention relates to compounds of the formula (I) or pharmaceutically acceptable salts thereof:

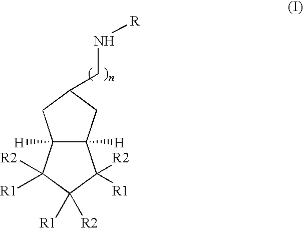

wherein:

R is selected from the group consisting of alkyl, cycloalkyl, haloalkyl, aryl, heteroaryl, aminocarbonyl alkyl, amide alkyl, heterocyclo aminocarbonyl alkyl and aminoalkyl, wherein the heterocycle is selected from the group consisting of 5- or 6-membered hetero ring further substituted, by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester and halogen;

R₁ and R₂ are each independently selected from the group consisting of hydrogen, hydroxyl, alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;

wherein R₁ and R₂ are attached together with the atom to form a 3 to 8 membered ring, wherein the 3 to 8 membered hetero ring further contains one or more heteroatoms selected from the group consisting of N, O and S, and the 3 to 8 membered hetero rings so formed are further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;

meanwhile, R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered rings so formed are further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₆ is selected from the group consisting of hydrogen or alkyl;

n is an integral from 0 to 4;

r is an integral from 1 to 6;

m is an integral from 0 to 6.

Further, the present invention includes the compounds of formula (Ia):

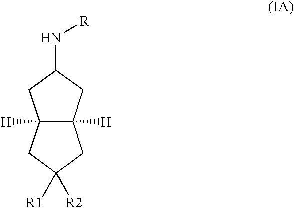

wherein:

R is selected from the group consisting of alkyl, cycloalkyl, haloalkyl, aryl, heteroaryl, aminocarbonyl alkyl, amide alkyl, heterocyclo aminocarbonyl alkyl and aminoalkyl, wherein the heterocycle is selected from the group consisting of 5- or 6-membered hetero ring further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester and halogen;

R₁ and R₂ are each independently selected from the group consisting of hydrogen, hydroxyl, alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;

wherein R₁ and R₂ are attached together with the atom to form a 3 to 8 membered ring, wherein the 3 to 8 membered hetero ring further contains one or more heteroatoms selected from the group consisting of N, O and S, and the 3 to 8 membered hetero rings so formed are each substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;

meanwhile, R₄ and R₅ are attached together the N atom to form the 3 to 8 membered hetero ring, wherein the 5 to 8 membered hetero ring further contains one or more heteroatoms selected from the group consisting of N, O and S, and the 3 to 8 membered hetero ring so formed are further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₆ is selected from the group consisting of hydrogen or alkyl;

r is an integral from 1 to 6;

m is an integral from 0 to 6.

Preferably, in the compounds of the formula (I) or pharmaceutically acceptable salts thereof,

R is heterocyclo aminocarbonyl alkyl, wherein the heterocycle is selected from the group consisting of 5- or 6-membered hetero ring further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester and halogen;

R₁ is hydrogen or hydroxyl; R₂ is selected from the group consisting of hydrogen, hydroxyl, alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkylamino, amide group, alkoxyl, aryloxyl, heterocylco alkyl, carboxylic acid and carboxylic ester;

R₃ is selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups selected from the group consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, carboxylic acid and carboxylic ester;

R₄ and R₅ are each independently selected from the group consisting of hydrogen, alkyl, cycloalkyl, aryl, heteroaryl and heterocyco alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;

meanwhile, R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered hetero rings so formed are substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₆ is selected from the group consisting of hydrogen or alkyl;

r is an integral from 1 to 6;

m is an integral from 0 to 6.

Preferably, in the compounds of the formula (I) or pharmaceutically acceptable salts thereof, R is the formula:

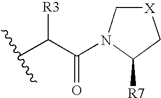

wherein R₃ is selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups selected from the group consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, carboxylic acid and carboxylic ester;

R₇ is selected from the group consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heteroalkyl, carboxylic acid, carboxylic ester and halogen;

X is C, S or O.

Preferably, n is 0.

In another aspect, this invention provides compounds of formula (I) or pharmaceutically acceptable salts, wherein the compounds of formula (I) are in the pharmaceutically acceptable free-form and the forms of acid addition salts, wherein the salts comprise the salts formed with the acids selected from the group consisting of hydrochloric acid, methanesulfonic acid, sulfuric acid, phosphoric acid, citric acid, acetic acid and trifluoroacetic acid, preferably, the acids are hydrochloric acid and trifluoroacetic acid.

This invention relates to the preparation process of the compounds of formula (I), wherein the preparation process comprises the following steps of:

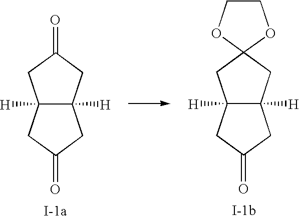

reacting starting material tetrahydro-pentalene-2,5-dione (I-1a) with ethylene glycol and a catalyst of p-toluenesulfonic acid through refluxing in the solvent of benzene to give 7,7-(ethylidene acetal)bicyclo[3.3.0] octane-3-one protected (I-1b);

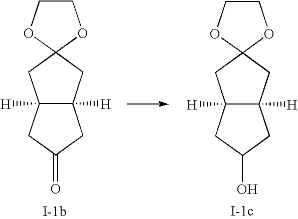

reducing the obtained 7,7-(ethylidene acetal)bicyclo[3.3.0] octane-3-one (I-1b) by NaBH₄ at room temperature to afford 3-hydroxy-7,7-(ethylidene acetal)bicyclo[3.3.0] octane (I-1c);

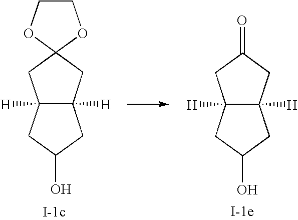

reacting the obtained 3-hydroxy-7,7-(ethylidene acetal)bicyclo[3.3.0] octane (I-1c) with oxalic acid in the mixed solvent of ethyl acetate and water to give 5-hydroxy-hexahydro-pentalen-2-one (I-1e);

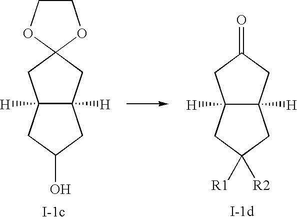

or reacting the obtained 3-hydroxy-7,7-(ethylidene acetal)bicyclo[3.3.0] octane (I-1c) with different isocyanate and trimethylchlorosilane at room temperature or with different Grignard reagent in the solvent of ether and then acidifying it further by 2N hydrochloric acid to give the compounds of the formula (I-1d);

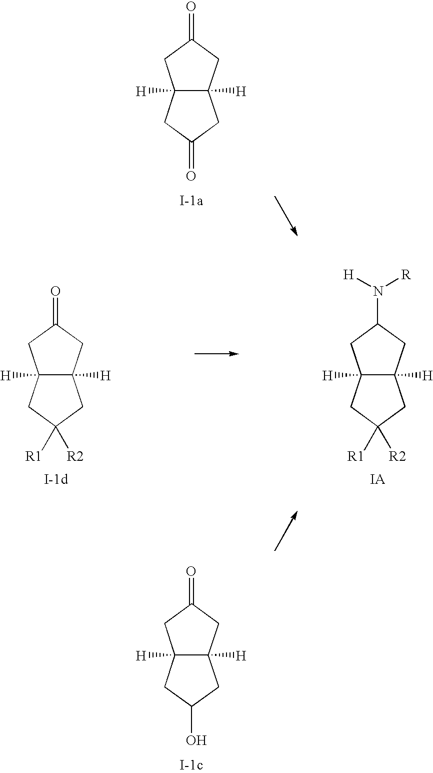

reacting the compounds of formula (I-1a) or formula (I-1d) or formula (I-1e) each independently with equivalent different amine in the solvent of methanol under the presence of sodium triethoxyborohydride and triethylamine at room temperature to give the compounds of formula (IA); wherein:

R is selected from the group consisting of alkyl, cycloalkyl, haloalkyl, aryl, heteroaryl, aminocarbonyl alkyl, amide alkyl, heterocyclo aminocarbonyl alkyl and aminoalkyl, wherein the heterocycle is selected from the group consisting of 5- or 6-membered hetero ring further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester and halogen;

R₁ and R₂ are each independently selected from the group consisting of hydrogen, hydroxyl, alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;

wherein R₁ and R₂ are attached together with the atom to form a 3 to 8 membered ring, wherein the 3 to 8 membered hetero ring further contains one or more heteroatoms selected from the group consisting of N, O and S, and the 3 to 8 membered hetero rings so formed are substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₃ is selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups selected from the group consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, carboxylic acid and carboxylic ester;

R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;

meanwhile, R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered hetero rings so formed are further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₆ is selected from the group consisting of hydrogen or alkyl;

r is an integral from 1 to 6;

m is an integral from 0 to 6.

Preferably, in the above described preparation process, wherein R is heterocyclo aminocarbonyl alkyl, wherein the heterocycle is selected from the group consisting of 5- or 6-membered hetero ring further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester and halogen;

R₁ is hydrogen or hydroxyl; R₂ is selected from the group consisting of hydrogen, hydroxyl, alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;

R₃ is selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups selected from the group consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, carboxylic acid and carboxylic ester;

R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;

meanwhile, R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered hetero rings so formed are substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₆ is selected from the group consisting of hydrogen or alkyl;

r is an integral from 1 to 6;

m is an integral from 0 to 6.

Preferably, R is the formula:

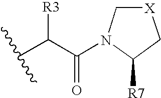

wherein, R₃ is selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups selected from the group consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, carboxylic acid and carboxylic ester;

R₇ is selected from the group consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heteroalkyl, carboxylic acid, carboxylic ester and halogen;

X is C, S or O.

Further, the above described preparation process also comprises that the obtain compounds of formula (IA) through purification are directly reacted with acid in the solvent of ether under an ice-water bath to give the acid addition salt thereof, or reacted with di-tert-butyl dicarbonate to protect nitrogen atom, purified the compounds by silica gel column chromatography, then reacted with acid in the solvent of ether under an ice-water bath to give the acid addition salt thereof, wherein the acids are hydrochloric acid, methanesulfonic acid, sulfuric acid, phosphoric acid, citric acid, acetic acid and trifluoroacetic acid, preferably, the acids are hydrochloric acid and trifluoroacetic acid.

Further, in the above described preparation process, wherein the compounds of formula (I-1a) or formula (I-1d) or formula (I-1e) is each independently reacted with equivalent RNH₂ in the solvent of methanol under the presence of sodium triethoxyborohydride and triethylamine at room temperature to give the compounds of formula (IA); wherein

R is selected from the group consisting of alkyl, cycloalkyl, haloalkyl, aryl, heteroaryl, aminocarbonyl alkyl, amide alkyl, heterocyclo aminocarbonyl alkyl and aminoalkyl, wherein the heterocycle is selected from the group consisting of 5- or 6-membered hetero ring further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl; carboxylic acid, carboxylic ester and halogen.

Preferably, R is the formula

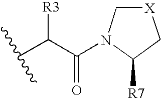

wherein:

R₃ is selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups selected from the group consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, carboxylic acid and carboxylic ester;

R₇ is selected from the group consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heteroalkyl, carboxylic acid, carboxylic ester and halogen;

X is C, S or O.

In a particularly preferred embodiment, the compounds of formula (I) or pharmaceutically acceptable salts consisting of:

| Example | | |
|---|---|---|
| No. | Structure | Name |
| 1 | 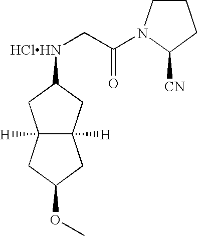 | 1-[2-(5-methoxy-octahydro- pentalen-2-ylamino)- acetyl]-pyrrolidine-2- carbonitrile hydrochloride |
| 2 | 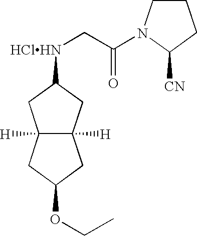 | 1-[2-(5-ethoxy-octahydro- pentalen-2-ylamino)- acetyl]-pyrrolidine-2- carbonitrile hydrochloride |
| 3 | 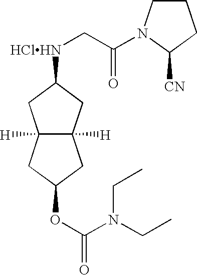 | 5-[2-(2-cyano-pyrrolidin- 1-yl)-2-oxo-ethylamino]- octahydro-pentalen-2- yl diethyl-carbamate hydrochloride |
| 4 | 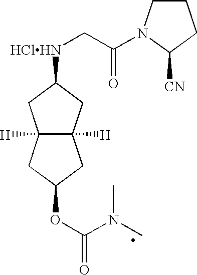 | 5-[2-(2-cyano-pyrrolidin- 1-yl)-2-oxo-ethylamino]- octahydro-pentalen-2-yl dimethyl-carbamate hydrochloride |
| 5 | 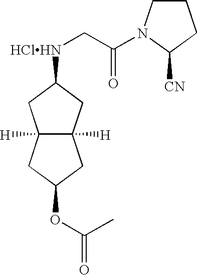 | 5-[2-(2-cyano-pyrrolidin- 1-yl)-2-oxo-ethylamino]- octahydro-pentalen-2-yl acetate hydrochloride |
| 6 | 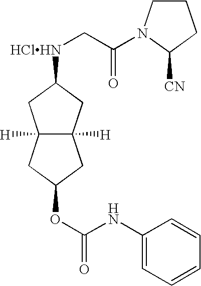 | 5-[2-(2-cyano-pyrrolidin- 1-yl)-2-oxo-ethylamino]- octahydro-pentalen-2-yl phenylcarbamate hydrochloride |
| 7 | 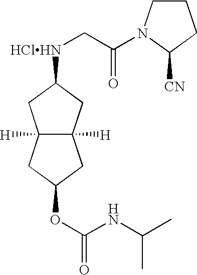 | 5-[2-(2-cyano-pyrrolidin- 1-yl)-2-oxo-ethylamino]- octahydro-pentalen-2-yl isopropyl-carbamate hydrochloride |
| 8 | 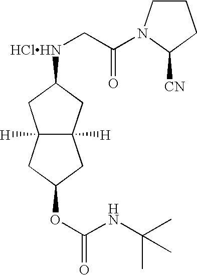 | 5-[2-(2-cyano-pyrrolidin- 1-yl)-2-oxo-ethylamino]- octahydro-pentalen-2-yl tert-butyl-carbamate hydrochloride |
| 9 | 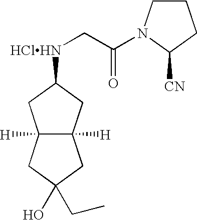 | 1-[2-(5-ethyl-5-hydroxy- octahydro-pentalen-2- ylamino)-1-hydroxy-ethyl]- pyrrolidine-2-carbonitrile hydrochloride acid |
| 10 | 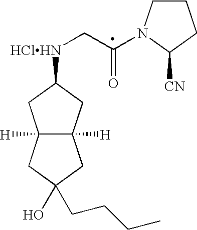 | 1-[2-(5-butyl-5-hydroxy- octahydro-pentalen-2-ylamino)-acetyl]- pyrrolidine-2-carbonitrile hydrochloride |
| 11 | 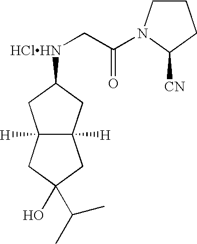 | 1-(2-(5-isopropyl-5- hydroxy-octahydro- pentalen-2-ylamino) acetyl) pyrrolidine-2-carbonitrile hydrochloride |
| 12 | 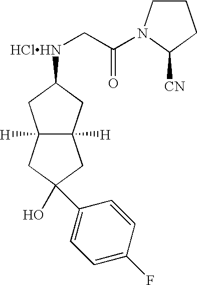 | 1-{2-[5-(4-fluoro-phenyl)- 5-hydroxy-octahydro- pentalen-2-ylamino]- acetyl}-pyrrolidine-2- carbonitrile hydrochloride |
| 13 | 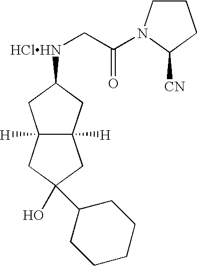 | 1-[2-(5-cyclohexyl-5- hydroxy-octahydro- pentalen-2-ylamino)-acetyl]- pyrrolidine-2-carbonitrile hydrochloride |
| 14 | 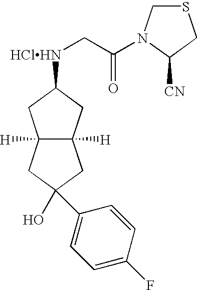 | 3-{2-[5-(4-fluoro-phenyl)- 5-hydroxy-octahydro- pentalen-2-ylamino]- acetyl}-thiazolidine-2- carbonitrile hydrochloride |
| 15 | 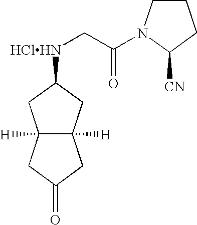 | 1-[2-(5-oxo-octahydro- pentalen-2-ylamino)- acetyl]-pyrrolidine-2- carbonitrile hydrochloride |
| 16 | 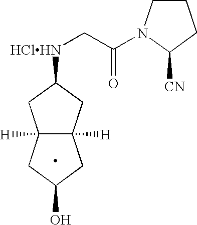 | 1-[2-(5-hydroxyl-octahydro- pentalen-2-ylamino)- acetyl]-pyrrolidine-2- carbonitrile hydrochloride |
| 17 | 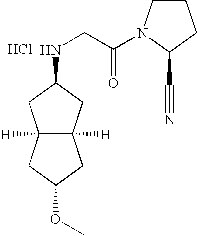 | 1-[2-(5-methoxy-octahydro- pentalen-2-ylamino)- acetyl]-pyrrolidine-2- carbonitrile hydrochloride |
| 18 | 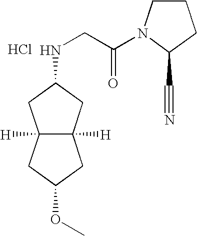 | 1-[2-(5-methoxy-octahydro- pentalen-2-ylamino)- acetyl]-pyrrolidine-2- carbonitrile hydrochloride |
| 19 | 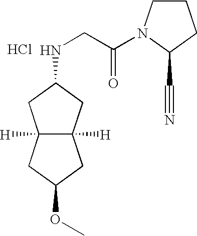 | 1-[2-(5 -methoxy-octahydro- pentalen-2-ylamino)- acetyl]-pyrrolidine-2- carbonitrile hydrochloride |

Further, this invention relates to compounds as intermediates in the synthesis of compounds of formula (I) having the following formula (I-1d) or (I-1e):

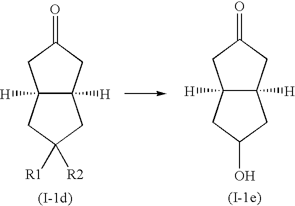

wherein:

R₁ and R₂ are each independently selected from the group consisting of alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ or —NR₄R₅, wherein the alkyl, cycloalkyl, heterocyclo alkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;

R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocycloalkoxyl, trifluoromethyl, carboxylic acid and carboxylic ester;

meanwhile, R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered hetero rings so formed are substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₆ is selected from the group consisting of hydrogen or alkyl;

r is an integral from 1 to 6;

m is an integral from 0 to 6.

Furthermore, this invention relates to the preparation process of compounds of formula (I-1d) or (I-1e), wherein the preparation process comprises the following steps of:

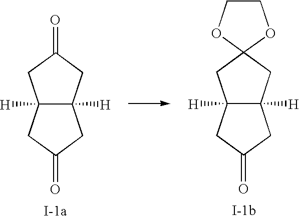

reacting starting material tetrahydro-pentalene-2,5-dione (I-1a) with ethylene glycol and a catalyst of p-toluenesulfonic acid through refluxing in the solvent of benzene to give 7,7-(ethylidene acetal)bicyclo[3.3.0] octane-3-one protected (I-1b);

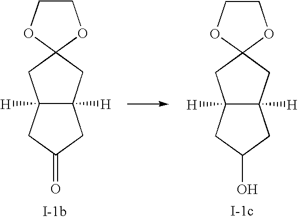

reducing the obtained 7,7-(ethylidene acetal)bicyclo[3.3.0] octane-3-one (I-1b) by NaBH₄ at room temperature to afford 3-hydroxy-7,7-(ethylidene acetal)bicyclo[3.3.0] octane (I-1c);

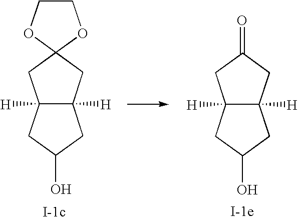

reacting the obtained 3-hydroxy-7,7-(ethylidene acetal)bicyclo[3.3.0] octane (I-1c) with oxalic acid in the mixed solvent of ethyl acetate and water to give 5-hydroxy-hexahydro-pentalen-2-one (I-1e);

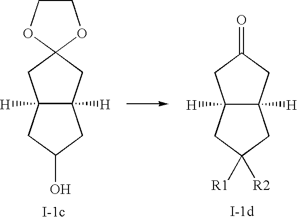

or reacting the obtained 3-hydroxy-7,7-(ethylidene acetal)bicyclo[3.3.0] octane (I-1c) with different isocyanate and trimethylchlorosilane at room temperature or with different Grignard reagent in the solvent of ether and then acidifying it further by 2N hydrochloric acid to give the compounds of the formula (I-1d).

In another aspect, this invention relates to a pharmaceutical composition comprising compounds or salts thereof of formula (I) in an effective therapeutic dose, as well as pharmaceutically acceptable carrier.

In another aspect, this invention relates to a use of the compounds or salts of formula (I) in the preparation of a medicament as a dipeptidyl peptidase IV inhibitor (DPPIV).

In other words, this invention is intended to provide the new bicyclooctane derivatives of formula (IB) and their tautomers, enantiomers, non-enantiomers, raceme, and pharmaceutically acceptable salts, and metabolites and metabolic precursors or prodrugs.

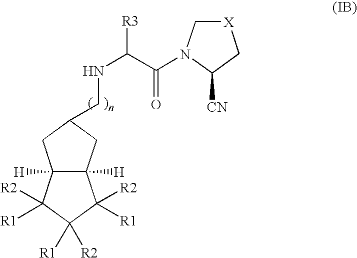

wherein:

X is C, S or O;

R₁ and R₂ are each independently selected from the group consisting of hydrogen, hydroxyl, alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocycloalkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;

wherein R₁ and R₂ are attached together with the atom to form a 3 to 8 membered ring, wherein the 3 to 8 membered hetero ring further contains one or more heteroatoms selected from the group consisting of N, O and S, and the 3 to 8 membered hetero rings so formed are substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₃ is selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups selected from the group consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, carboxylic acid and carboxylic ester;

R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;

meanwhile, R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered hetero rings so formed are substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₆ is selected from the group consisting of hydrogen or alkyl;

n is an integral from 0 to 4;

r is an integral from 1 to 6;

m is an integral from 0 to 6.

Preferably, this invention relates to compounds or pharmaceutically acceptable salts of formula (IC):

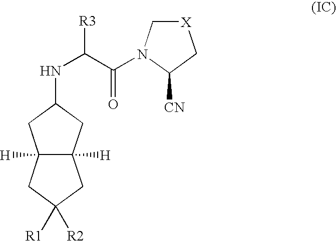

X is C, S or O;

R₁ and R₂ are each independently selected from the group consisting of hydrogen, hydroxyl, alkyl, cycloalkyl, heterocyclo alkyl, aryl, heteroaryl, —OR₄, —(CH₂CH₂O)ᵣR₆, —(CH₂)ₘC(O)OR₄, (CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;

wherein R₁ and R₂ are attached together with the atom to form a 3 to 8 membered ring, wherein the 3 to 8 membered hetero ring further contains one or more heteroatoms selected from the group consisting of N, O and S, and the 3 to 8 membered hetero rings so formed are further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₃ is selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups selected from the group consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, carboxylic acid and carboxylic ester;

R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;

meanwhile, R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered rings so formed are further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₆ is selected from the group consisting of hydrogen or alkyl;

r is an integral from 1 to 6;

m is an integral from 0 to 6.

This invention also relates to compounds as intermediates in the synthesis of compounds of formula (IB) having the following formula (I-1d) or (I-1e):

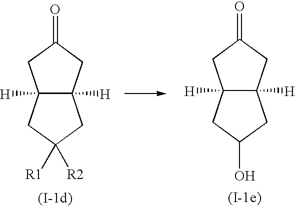

wherein:

R₁ and R₂ are each independently selected from the group consisting of alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;

R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;

meanwhile, R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered hetero rings so formed are substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen or —NR₄R₅;

R₆ is selected from the group consisting of hydrogen or alkyl;

r is an integral from 1 to 6; and

m is an integral from 0 to 6.

This invention relates to compounds of formula (IB) or pharmaceutically acceptable salts, preferably:

R₁ is hydrogen or hydroxyl, R₂ is selected from the group consisting of hydrogen, hydroxyl, alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkylamino, amide group, alkoxyl, aryloxyl, heterocylco alkyl, carboxylic acid and carboxylic ester;

R₃ is selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups selected from the group consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, carboxylic acid and carboxylic ester;

R₄ and R₅ are independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid or carboxylic ester;

meanwhile, R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered hetero rings so formed are substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;

R₆ is selected from the group consisting of hydrogen or alkyl;

r is an integral from 1 to 6; and

m is an integral from 0 to 6.

This invention relates to compounds of formula (IB) or pharmaceutically acceptable salts, preferably, X is C, S or O.

This invention relates to compounds of formula (IB) or pharmaceutically acceptable salts, preferably, n is 0.

A preparation process of compounds of formula (IC) or pharmaceutically acceptable salts, wherein the compounds of formula (I-1a), formula (I-1d) or formula (I-1e) are each independently reacted with different amine in the solvent of methanolunder the presence of sodium triethoxyborohydride and triethylamine at room temperature to give the compounds of formula (IC);

This invention relates to compounds of formula (IB) or pharmaceutically acceptable salts, wherein the compounds of formula (IB) are in pharmaceutically acceptable free-form and the forms of acid addition salts, and provide the pharmaceutically acceptable (nontoxic, physiologically acceptable) salts thereof;

wherein the pharmaceutically acceptable salts are selected from the group consisting of hydrochloride, methanesulfonate, sulfate, phosphate, citrate, acetate and trifluoroacetate. Preferably, the salts are hydrochloride and trifluoroacetate. More preferably, the salts are hydrochloride.

### Synthesis Method of the Invention Compound

In order to complete the objection of the invention, the invention applies the following technical solution:

A preparation process of compounds of formula (IC) or pharmaceutically acceptable salts of the invention, comprising the following steps of:

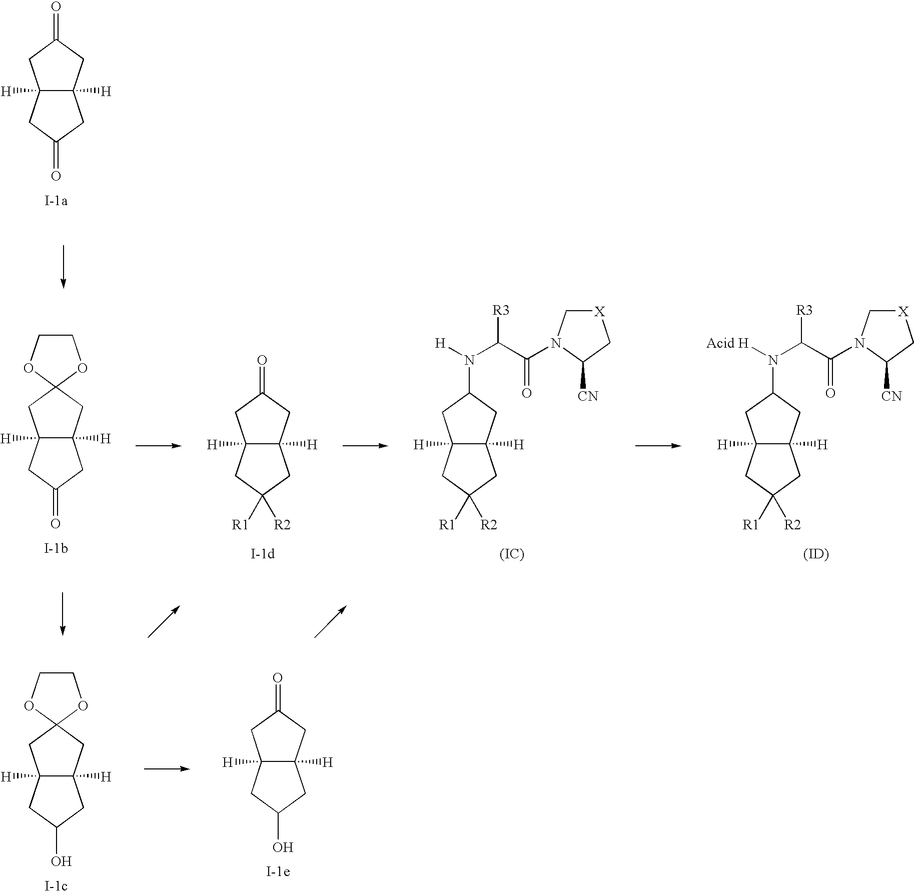

reacting starting material tetrahydro-pentalene-2,5-dione (I-1a) with ethylene glycol and a catalyst of p-toluenesulfonic acid through refluxing in the solvent of benzene to give 7,7-(ethylidene acetal)bicyclo[3.3.0] octane-3-one protected (I-1b); then reducing the obtained 7,7-(ethylidene acetal)bicyclo[3.3.0] octane-3-one (I-1b) by NaBH₄ at room temperature to afford 3-hydroxy-7,7-(ethylidene acetal)bicyclo[3.3.0] octane (I-1c); reacting the obtained 3-hydroxy-7,7-(ethylidene acetal)bicyclo[3.3.0] octane (I-1c) with oxalic acid in the mixed solvent of ethyl acetate and water to give 5-hydroxy-hexahydro-pentalen-2-one (I-1e); or reacting the obtained 3-hydroxy-7,7-(ethylidene acetal)bicyclo[3.3.0] octane (I-1c) with different isocyanate and trimethylchlorosilane at room temperature or with different Grignard reagent in the solvent of ether and then acidifying it further by 2N hydrochloric acid to give the compounds of the formula (I-1d); reacting the compounds of formula (I-1a) or formula (I-1d) or formula (I-1e) each independently with equivalent different amine in the solvent of methanol under the presence of sodium triethoxyborohydride and triethylamine at room temperature to give the compounds of formula (IC); reacting the compounds of formula (IC) through purification directly with acid in the solvent of ether under an ice-water bath to give the acid addition salts thereof, or reacting the compounds with di-tert-butyl dicarbonate to protect nitrogen atom, purifying the compounds by silica gel column chromatography, then reacting the compounds with acid in the solvent of ether under an ice-water bath to give the acid addition salts thereof.

This invention relates to a pharmaceutical composition comprising a compound or salt in an effective therapeutic dose, as well as pharmaceutically acceptable carrier, or this invention relates to a use of the compounds or salts in the preparation of a medicament as a dipeptidyl peptidase IV inhibitor. In other words, this invention also provides the composition comprising the above compound in an effective therapeutic dose, and the use of the compounds in the preparation of a medicament as a dipeptidyl peptidase IV inhibitor.

### Specific Implementation Methods

The following examples serve to illustrate the invention, but the examples should not be considered as limiting the scope of the invention.

### EXAMPLES

The compound's structure determination was confirmed by NMR and MS. NMR chemical shifts were given in ppm (10⁻⁶). NMR is determined by a Bruker AVANCE-400 machine. The solvent were deuterated-chloroform (CDCl₃) and deuterated-dimethyl sulfoxide (DMSO-d6) with tetramethylsilane (TMS) as internal standard. Chemical shifts were given in ppm (10⁻⁶).

MS is determined by a FINNIGA N LCQ Ad (ESI) mass spectrometer.

IC₅₀ is determined by a NovoStar ELIASA (BMG Co. German).

Thin-layer silica gel is yantai huanghai HSGF254 or qingdao GF254 silica gel plate.

Column chromatography generally uses yantai huanghai 200˜300 mesh silica gel as carrier.

DMSO-D₆: deuterated-dimethyl sulfoxide;

CDCl₃: deuterated-chloroform.

### Example 1

### Preparation of 1-[2-(5-methoxy-octahydro-pentalen-2-ylamino)-acetyl]-pyrrolidine-2-carbonitrile Hydrochloride 1

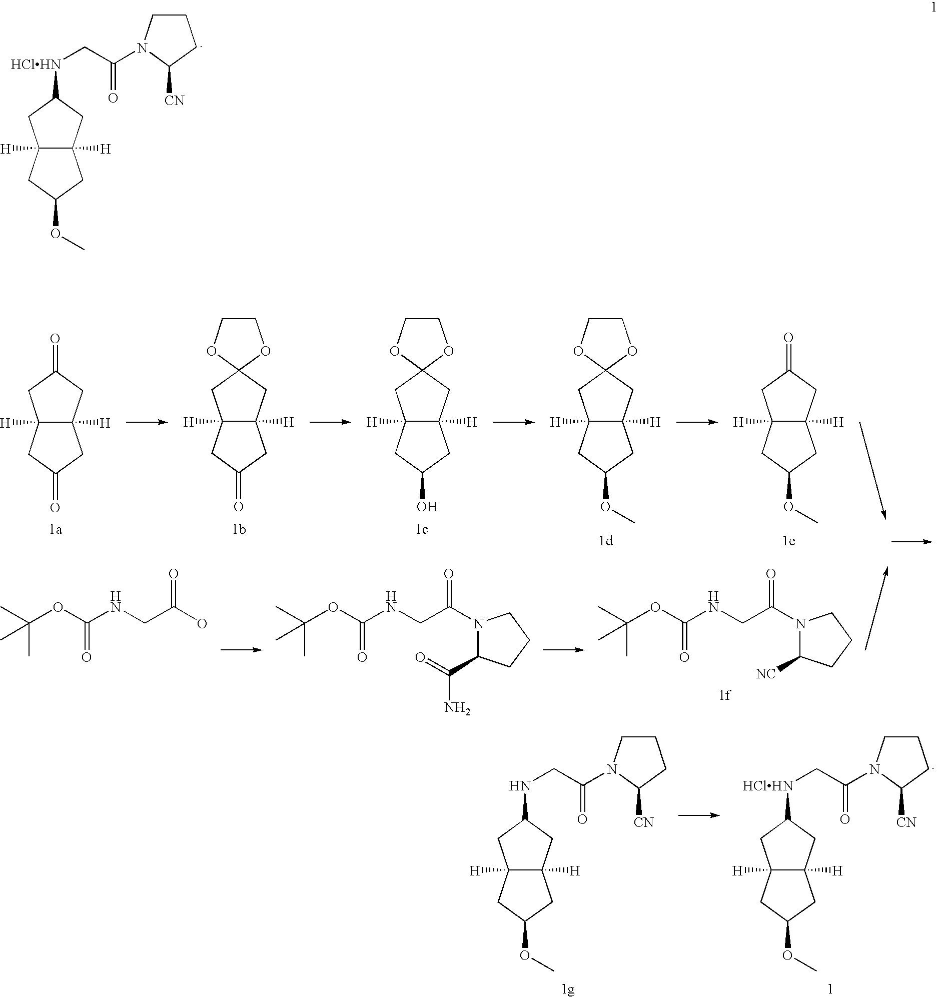

### Preparation of 7,7-(ethylidene acetal)bicyclo[3.3.0] octane-3-one 1b

In a 250 ml round bottom flask equipped with a water segregator, tetrahydro-pentalene-2,5-dione 1a (3 g, 21 mmol) and ethane-1,2-diol (1.03 mL, 18.5 mmol) were dissolved in 150 mL benzene under stirring, then 4-methylbenzenesulfonic acid (50 mg, 0.21 mmol) was added. Upon completion of the addition, the reaction mixture was heated to reflux overnight and then cooled to room temperature. The solvent of benzene was removed under reduced pressure. The residue was purified by silica gel column chromatography to give the title compound 7,7-(ethylidene acetal)bicyclo[3.3.0] octane-3-one 1b (1.8 g, yield 56%) as a colorless oil.

MS m/z (ESI): 183.5 [M+1].

### Preparation of 3-hydroxy-7,7-(ethylidene acetal)bicyclo[3.3.0] Octane 1c

7,7-(Ethylidene acetal)bicyclo[3.3.0] octane-3-one 1b (1 g, 5.5 mmol) was dissolved in 15 mL methanol, sodium borohydride (0.4 g, 11 mmol) in batch was added in an ice-water bath. Upon completion of the addition, the reaction mixture was stirred at room temperature for 1-2 hours. The reaction was stopped by adding acetone. The mixture was extracted with ethyl acetate (50 mL×3). The combined organic phase was washed with 50 mL saturated brine, dried over anhydrous sodium sulfate, filtered and concentrated under reduced pressure to give the title compound 3-hydroxy-7,7-(ethylidene acetal)bicyclo[3.3.0] octane 1c (1.01 g, yield 99.9%) as a colorless oil.

MS m/z (ESI): 185.2 [M+1].

### Preparation of 3-methoxy-7,7-(ethylidene acetal)bicyclo[3.3.0] Octane 1d

3-Hydroxy-7,7-(ethylidene acetal)bicyclo[3.3.0] octane 1c (1 g, 5.4 mmol) was dissolved in 30 mL tetrahydrofuran, then sodium hydride (522 mg, 10.9 mmol) was added under stirring. Upon completion of the addition, the reaction mixture was stirred for 1 hour at room temperature, then added with methyl iodide (1.35 mL, 21.7 mmol) and heated to reflux for 3 hours. The reaction was stopped by adding ice-water, the reaction mixture was extracted with ethyl acetate (30 mL×3). The combined organic phase was washed with 30 mL saturated brine, dried over anhydrous sodium sulfate, filtered and concentrated under reduced pressure. The residue was purified by silica gel column chromatography to give the title compound 3-methoxy-7,7-(ethylidene acetal)bicyclo[3.3.0] octane 1d (944 mg, yield 88%) as a colorless oil.

MS m/z (ESI): 199.6 [M+1].

### Preparation of 5-methoxy-hexahydro-pentalen-2-one 1e

3-Methoxy-7,7-(ethylidene acetal)bicyclo[3.3.0] octane 1d (994 mg, 5.02 mmol) was dissolved in the mixture of 30 mL ethyl acetate and 10 mL water, then ethanedioic acid (632 mg, 5.02 mmol) was added under stirring. Upon completion of the addition, the reaction mixture was stirred at room temperature until thin lay chromatography (TLC) showed the starting material disappeared, the reaction mixture was extracted with ethyl acetate (30 mL×3). The combined organic phase was washed with 30 mL saturated brine, dried over anhydrous sodium sulfate, filtered and concentrated under reduced pressure. The residue was purified by silica gel column chromatography to give the title compound 5-methoxy-hexahydro-pentalen-2-one 1e (610 mg, yield 79%) as a light yellow oil.

MS m/z (ESI): 155.1 [M+1].

### Preparation of [2-(2-cyano-pyrrolidin-1-yl)-2-oxo-ethyl]-carbamic Acid tert-butyl Ester 1f

1) Preparation of [2-(2-carbamoyl-pyrrolidin-1-yl)-2-oxo-ethyl]-carbamic Acid tert-butyl Ester

N-tert-butyloxycarbonyl glycine (5 g, 28.56 mmol) and L-pyrrolidine-2-carboxamide (3.25 g, 28.50 mmol) were dissolved in 75 mL N,N-dimethylformamide at 0°, then 1-hydroxybenzotriazole (11.8 g, 87.3 mmol) and N-ethyl-N′-(dimethylaminopropyl)-carbodiimide (11.3 g, 59 mmol) and triethylamine (12.1 mL, 87.3 mmol) were adding under stirring. Upon completion of the addition, the reaction mixture was naturally raised to room temperature, stirred overnight. The resulting mixture was concentrated under reduced pressure below 50°, extracted with ethyl acetate (200 mL×3). The combined organic phase was washed with 50 mL saturated brine, dried over anhydrous sodium sulfate, filtered and concentrated under reduced pressure. The residue was purified by recrystallization with ethyl acetate to give the title compound [2-(2-carbamoyl-pyrrolidin-1-yl)-2-oxo-ethyl]-carbamic acid tert-butyl ester 1f (7.42 g, yield 95.8%) as a white powder.

MS m/z (ESI): 272.1 [M+1].

2) Preparation of [2-(2-cyano-pyrrolidin-1-yl)-2-oxo-ethyl]-carbamic Acid tert-butyl Ester 1f

In dry three-neck flask under a nitrogen atmosphere, 286 mL pyridine, [2-(2-carbamoyl-pyrrolidin-1-yl)-2-oxo-ethyl]-carbamic acid tert-butyl ester (13.5 g, 49.8 mmol) and imidazole (7.11 g, 104.6 mmol) were added in order, phosphorus oxychloride (19 mL, 204.2 mmol) was added at −35° under stirring. Upon completion of the addition, the reaction mixture was stirred for 1 hour at −35°, then naturally raised to room temperature and reacted for 0.5 hour. Pyridine was evaporated and the reaction mixture was extracted with ethyl acetate (200 mL×3). The combined organic phase was washed with 50 mL saturated brine, dried over anhydrous sodium sulfate, filtered and concentrated under reduced pressure. The residue was purified by silica gel column chromatography to give the title compound [2-(2-cyano-pyrrolidin-1-yl)-2-oxo-ethyl]-carbamic acid tert-butyl ester 1f (10.7 g, yield 84.9%) as a white powder.

MS m/z (ESI): 254.3 [M+1].

### Preparation of 1-[2-(5-methoxy-octahydro-pentalen-2-ylamino)-acetyl]pyrrolidine-2-carbonitrile 1g

[2-(2-Cyano-pyrrolidin-1-yl)-2-oxo-ethyl]-carbamic acid tert-butyl ester 1f (740 mg, 2.92 mmol) was dissolved in 20 mL dichloromethane, then trifluoroacetic acid (6.71 mL, 87.6 mmol) was added in an ice-water bath. Upon completion of the addition, the reaction mixture was stirred at 0° until the reaction was completed. Dichloromethane and trifluoroacetic acid were evaporated. The above residue was dissolved in 20 mL methanol, in order added with triethylamine (0.813 mL, 5.844 mmol), 5-methoxy-hexahydro-pentalen-2-one 1e (310 mg, 1.948 mmol) and sodium triacetoxy borohydride (1.65 g, 7.792 mmol). Upon completion of the addition, the reaction mixture was stirred overnight at room temperature, concentrated under reduced pressure, added with 20 mL saturated sodium carbonate solution, extracted with dichloromethane (20 mL×3). The combined organic phase was washed with 10 mL saturated brine, the dichloromethane phase was dried over anhydrous sodium sulfate, filtered and concentrated under reduced pressure. The residue was purified by silica gel column chromatography to give the title compound 1-[2-(5-methoxy-octahydro-pentalen-2-ylamino)-acetyl]pyrrolidine-2-carbonitrile 1g (200 mg, yield 35.3%) as a white powder.

MS m/z (ESI): 292.7 [M+1].

¹H NMR (400 MHz, CDCl₃) δ (ppm) 4.79 (m, 1H), 3.82 (m, 2H), 3.67 (m, 2H), 3.48 (m, 1H), 3.26 (s, 3H), 2.40 (m, 2H), 2.36-1.98 (m, 6H), 1.85 (m, 3H), 1.62 (m, 2H), 1.53-1.14 (m, 2H).

### Preparation of 1-[2-(5-methoxy-octahydro-pentalen-2-ylamino)-acetyl]pyrrolidine-2-carbonitrile Hydrochloride 1

1-[2-(5-Methoxy-octahydro-pentalen-2-ylamino)-acetyl]pyrrolidine-2-carbonitrile 1g (200 mg, 0.687 mmol) was dispersed in 10 mL ether, a solution of 0.5N hydrochloric acid in 2 mL ether was added in an ice-water bath. The resulting solid was centrifuged to give the title compound 1-[2-(5-methoxy-octahydro-pentalen-2-ylamino)-acetyl]pyrrolidine-2-carbonitrile hydrochloride 1 (180 mg, yield 80%) as a white powder.

MS m/z (ESI): 292.7 [M+1].

### Example 2

### Preparation of 1-[2-(5-ethoxy-octahydro-pentalen-2-ylamino)-acetyl]-pyrrolidine-2-carbonitrile Hydrochloride 2

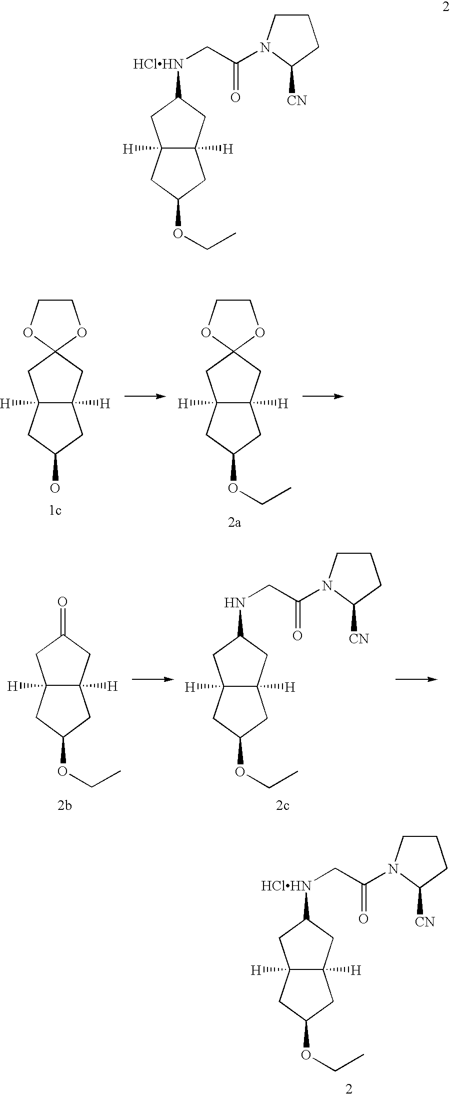

### Preparation of 3-ethoxyl-7,7-(ethylidene acetal)bicyclo[3.3.0] Octane 2a

3-Hydroxy-7,7-(ethylidene acetal)bicyclo[3.3.0] octane 1c (1 g, 5.4 mmol) was dissolved in 20 mL tetrahydrofuran, 50% sodium hydride (521 mg) was added under stirring. Upon completion of the addition, the reaction mixture was stirred for 1 hour, then added with ethyl iodide (0.869 mL, 10.86 mmol) and heated to reflux for 3 hours. The reaction was stopped by adding ice-water, the reaction mixture was extracted with ethyl acetate (80 mL×3). The combined organic phase was washed with 15 mL saturated brine, the ethyl acetate phase was dried over anhydrous sodium sulfate, filtered and concentrated under reduced pressure. The residue was purified by silica gel column chromatography to give the title compound 3-ethoxyl-7,7-(ethylidene acetal)bicyclo[3.3.0] octane 2a (1.15 g, yield 100%) as a light yellow oil.

MS m/z (ESI): 213.2 [M+1].

### Preparation of 5-ethoxy-hexahydro-pentalen-2-one 2b

3-Ethoxyl-7,7-(ethylidene acetal)bicyclo[3.3.0] octane 2a (1.15 g, 5.4 mmol) was dissolved in the mixture of 30 mL ethyl acetate and 10 mL water, then ethanedioic acid (1.368 g, 10.85 mmol) was added under stirring. Upon completion of the addition, the reaction mixture was stirred at room temperature until TLC showed the starting material 2a disappeared, and the reaction mixture was extracted with ethyl acetate (50 mL×3). The combined organic phase was washed with 50 mL saturated brine, the ethyl acetate phase was dried over anhydrous sodium sulfate, filtered and concentrated under reduced pressure. The residue was purified by silica gel column chromatography to give the title compound 5-ethoxy-hexahydro-pentalen-2-one 2b (871 mg, yield 80%) as a white powder.

MS m/z (ESI): 169.3 [M+1].

### Preparation of 1-[2-(5-ethoxy-octahydro-pentalen-2-ylamino)-acetyl]-pyrrolidine-2-carbonitrile 2c

[2-(2-Cyano-pyrrolidin-1-yl)-2-oxo-ethyl]-carbamic acid tert-butyl ester 1f (903 mg, 3.57 mmol) was dissolved in 20 mL dichloromethane in an ice-water bath, trifluoroacetic acid (8.2 mL, 107.1 mmol) was added. Upon completion of the addition, the reaction mixture was stirred at 0° until the reaction was completed. Dichloromethane and trifluoroacetic acid were evaporated. The above residue was dissolved in 20 mL methanol, in order added with triethylamine (0.993 mL, 7.143 mmol), 5-ethoxy-hexahydro-pentalen-2-one 2b (400 mg, 2.38 mmol) and sodium triacetoxy borohydride (2.017 g, 9.52 mmol) at room temperature. The reaction mixture was concentrated and added with 50 mL saturated sodium carbonate solution, then extracted with dichloromethane (50 mL×3). The combined organic phase was washed with 50 mL saturated brine, the dichloromethane phase was dried over anhydrous sodium sulfate, filtered and concentrated under reduced pressure. The residue was purified by column chromatography to give the title compound 1-[2-(5-ethoxy-octahydro-pentalen-2-ylamino)-acetyl]-pyrrolidine-2-carbonitrile 2c (182 mg, yield 30%) as a white powder.

MS m/z (ESI): 306.5 [M+1].

¹H NMR (400 MHz, CDCl₃) δ (ppm) 4.78 (m, 1H), 3.89 (m, 2H), 3.65 (m, 2H), 3.44 (m, 3H), 2.38 (m, 2H), 2.32 (m, 2H), 2.22 (m, 2H), 2.13 (m, 2H), 1.89-1.82 (m, 2H), 1.58 (m, 3H), 1.32-1.26 (m, 2H), 1.15 (m, 3H).

### Preparation of 1-[2-(5-ethoxy-octahydro-pentalen-2-ylamino)-acetyl]-pyrrolidine-2-carbonitrile Hydrochloride 2

1-[2-(5-Ethoxy-octahydro-pentalen-2-ylamino)-acetyl]-pyrrolidine-2-carbonitrile 2c (182 mg, 0.598 mmol) was dispersed in 15 mL ether, a solution of 0.5N hydrochloric acid in 3 mL ether was added in an ice-water bath. The resulting solid was centrifuged to give the title compound 1-[2-(5-ethoxy-octahydro-pentalen-2-ylamino)-acetyl]-pyrrolidine-2-carbonitrile hydrochloride 2 (160 mg, yield 80%) as a white powder.

MS m/z (ESI): 306.4 [M+1].

### Example 3

### Preparation of 5-[2-(2-cyano-pyrrolidin-1-yl)-2-oxo-ethylamino]-octahydro-pentalen-2-yl diethyl-carbamate Hydrochloride 3

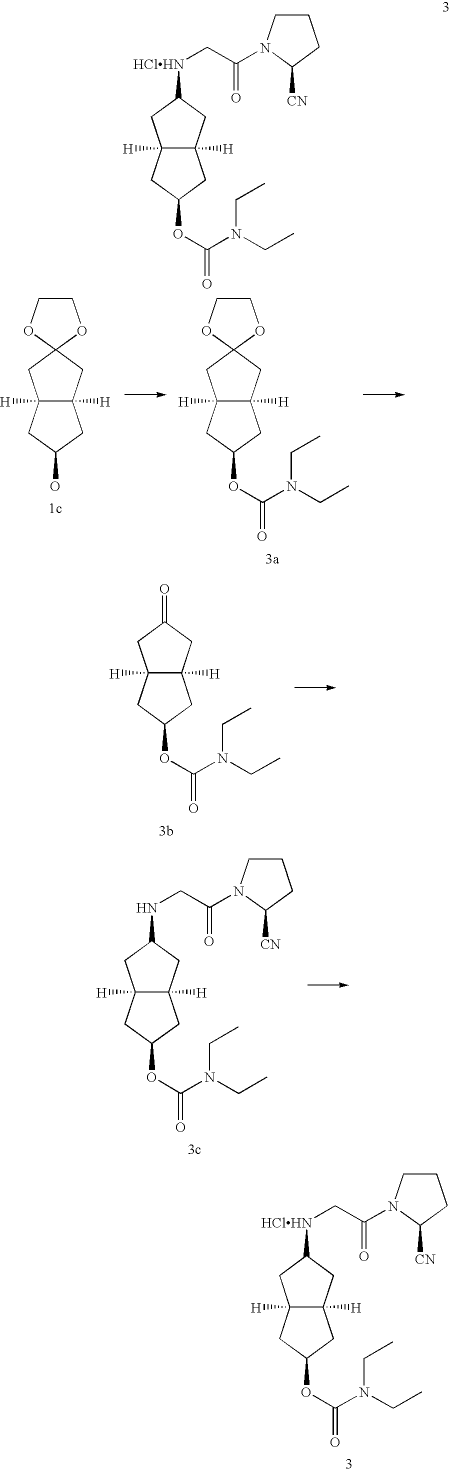

### Preparation of 5,5-(ethylidene acetal)-octahydro-pentalen-2-yl diethyl-carbamate 3a

Under an argon atmosphere, 3-hydroxy-7,7-(ethylidene acetal)bicyclo[3.3.0] octane 1c (1.3 g, 7.065 mmol) was dissolved in 18 mL pyridine, then 4-dimethylamino pyridine (0.26 g, 2.12 mmol) was added, diethylamino formyl chloride (2.68 mL, 21.20 mmol) was added dropwise under stirring. Upon completion of the addition, the reaction mixture was heated to reflux overnight. Pyridine was evaporated after the reaction was completed, the reaction mixture was extracted with ethyl acetate (50 mL×3). The combined organic phase was washed with 50 mL saturated brine, the ethyl acetate was dried over anhydrous sodium sulfate, filtered and concentrated under reduced pressure. The residue was purified by silica gel column chromatography to give the title compound 5,5-(ethylidene acetal)-octahydro-pentalen-2-yl diethyl-carbamate 3a (1.57 g, yield 88%) as a light yellow oil.

MS m/z (ESI): 284.3 [M+1].

### Preparation of 5-oxo-octahydro-pentalen-2-yl diethyl-carbamate 3b

5,5-(Ethylidene acetal)-octahydro-pentalen-2-yl diethyl-carbamate 3a (140 mg, 4.947 mmol) was dissolved in the mixture of 60 mL ethyl acetate and 15 mL water, then ethanedioic acid (623 mg, 4.947 mmol) was added under stirring. Upon completion of the addition, the reaction mixture was stirred at room temperature until TLC showed the starting material 3a disappeared, and the reaction mixture was extracted with ethyl acetate (60 mL×3). The combined organic phase was washed with 50 mL saturated brine, the ethyl acetate phase was dried over anhydrous sodium sulfate, filtered and concentrated under reduced pressure. The residue was purified by silica gel column chromatography to give the title compound 5-oxo-octahydro-pentalen-2-yl diethyl-carbamate 3b (1.02 g, yield 86%) as a white powder.

MS m/z (ESI): 240.5 [M+1].

### Preparation of 5-[2-(2-cyano-pyrrolidin-1-yl)-2-oxo-ethylamino]-octahydro-pentalen-2-yl diethyl-carbamate 3c

[2-(2-Cyano-pyrrolidin-1-yl)-2-oxo-ethyl]-carbamic acid tert-butyl ester 1f (530 mg, 2.1 mmol) was dissolved in 20 mL dichloromethane, then trifluoroacetic acid (4.8 mL, 63 mmol) was added under stirring in an ice-water bath. Upon completion of the addition, the reaction mixture was stirred at 0° until the reaction was completed. Dichloromethane and trifluoroacetic acid were evaporated. The above residue was dissolved in 20 mL methanol, in order added with triethylamine (0.88 mL, 6.3 mmol), 5-oxo-octahydro-pentalen-2-yl diethyl-carbamate 3b (500 mg, 2.1 mmol) and sodium triacetoxy borohydride (1.34 g, 6.3 mmol). The reaction mixture was stirred overnight at room temperature, concentrated and added with 50 mL saturated sodium carbonate solution, extracted with dichloromethane (50 mL×3). The combined organic phase was washed with 50 mL saturated brine, the dichloromethane was dried over anhydrous sodium sulfate, filtered and concentrated under reduced pressure. The residue was purified by silica gel column chromatography to give the title compound 5-[2-(2-cyano-pyrrolidin-1-yl)-2-oxo-ethylamino]-octahydro-pentalen-2-yl diethyl-carbamate 3c (400 mg, yield 50%) as a white powder.

MS m/z (ESI): 377.2 [M+1].

¹H NMR (400 MHz, CDCl₃) δ (ppm) 5.07 (m, 1H), 4.79 (m, 1H), 3.92-3.40 (m, 4H), 3.25 (m, 4H), 2.39 (m, 2H), 2.27 (m, 2H), 2.20-2.12 (m, 6H), 1.69 (m, 1H), 1.57-1.54 (m, 2H), 1.50-1.23 (m, 2H), 1.1 (m, 6H).

### Preparation of 5-[2-(2-cyano-pyrrolidin-1-yl)-2-oxo-ethylamino]-octahydro-pentalen-2-yl diethyl-carbamate Hydrochloride 3

5-[2-(2-Cyano-pyrrolidin-1-yl)-2-oxo-ethylamino]-octahydro-pentalen-2-yl diethyl-carbamate 3c (200 mg, 0.53 mmol) was dispersed in 15 mL ether, a 3 mL solution of 0.5N hydrochloric acid in ether was added in an ice-water bath. The resulting solid was centrifuged to give the title compound 5-[2-(2-cyano-pyrrolidin-1-yl)-2-oxo-ethylamino]-octahydro-pentalen-2-yl diethyl-carbamate hydrochloride 3 (180 mg, yield 82%) as a white powder.

MS m/z (ESI): 377.3 [M+1].

### Example 4

### Preparation of 5-[2-(2-cyano-pyrrolidin-1-yl)-2-oxo-ethylamino]-octahydro-pentalen-2-yl dimethyl-carbamate Hydrochloride 4

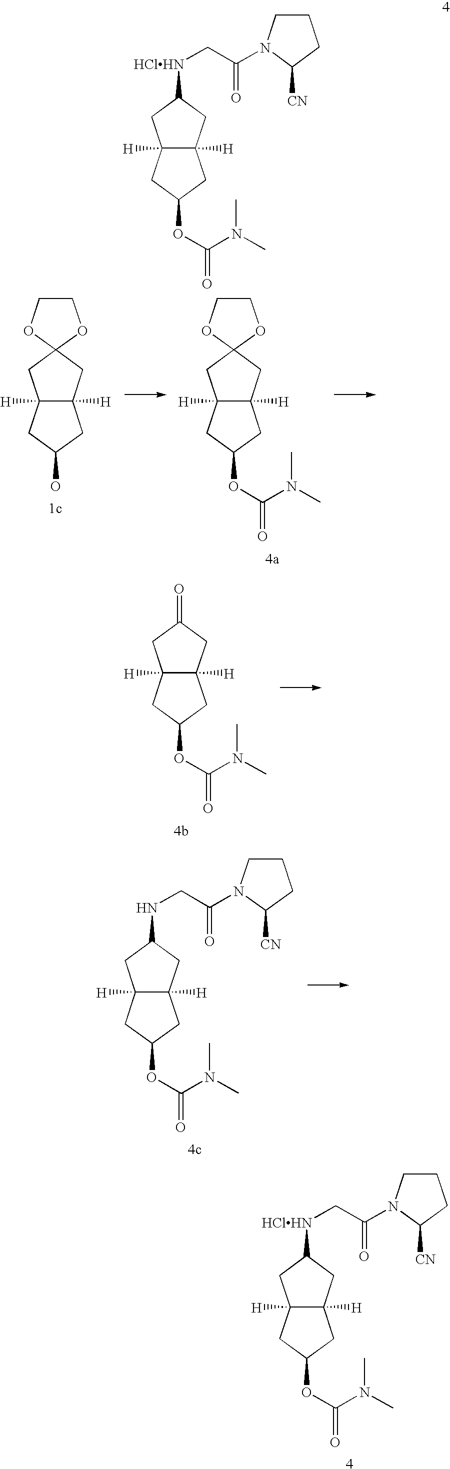

### Preparation of 5,5-(ethylidene acetal)-octahydro-pentalen-2-yl dimethyl-carbamate 4a

Under an argon atmosphere, 3-hydroxy-7,7-(ethylidene acetal)bicyclo[3.3.0]octane 1c (1.3 g, 7.065 mmol) was dissolved in 15 mL pyridine, then 4-dimethylamino pyridine (0.26 g, 2.120 mmol) was added, dimethylamino formyl chloride (1.95 mL, 21.20 mmol) was added dropwise under stirring. Upon completion of the addition, the reaction mixture was heated to reflux overnight. Pyridine was evaporated after the reaction was completed, the reaction mixture was extracted with ethyl acetate (50 mL×3). The combined organic phase was washed with 15 mL saturated brine, the ethyl acetate phase was dried over anhydrous sodium sulfate, filtered and concentrated under reduced pressure. The residue was purified by silica gel column chromatography to give the title compound 5,5-(ethylidene acetal)-octahydro-pentalen-2-yl dimethyl-carbamate 4a (1.8 g, yield 67%) as a light yellow oil.

MS m/z (ESI): 256.0 [M+1].

### Preparation of 5-oxo-octahydro-pentalen-2-yl dimethyl-carbamate 4b

In an ice-water bath, dimethyl-carbamic acid 5,5-(ethylidene acetal)-octahydro-pentalen-2-yl ester 4a (830 mg, 3.255 mmol) was dissolved in the mixture of ethyl acetate (60 mL) and water (10 mL), then ethanedioic acid (410 mg, 3.255 mmol) was added under stirring. Upon completion of the addition, the reaction mixture was stirred at room temperature until TLC showed the starting material 4a disappeared, and the reaction mixture was extracted with ethyl acetate (50 mL×3). The combined organic phase was washed with 15 mL saturated brine, the ethyl acetate phase was dried over anhydrous sodium sulfate, filtered and concentrated under reduced pressure. The residue was purified by silica gel column chromatography to give the title compound dimethyl-carbamic acid 5-oxo-octahydro-pentalen-2-yl ester 4b (600 mg, yield 87%) as a light yellow oil.

MS m/z (ESI): 212.2 [M+1].

### Preparation of 5-[2-(2-cyano-pyrrolidin-1-yl)-2-oxo-ethylamino]-octahydro-pentalen-2-yl dimethyl-carbamate 4c

[2-(2-Cyano-pyrrolidin-1-yl)-2-oxo-ethyl]-carbamic acid tert-butyl ester 1f (835 mg, 3.3 mmol) was dissolved in 20 mL dichloromethane, then trifluoroacetic acid (7.66 mL, 100 mmol) was added under stirring in an ice-water bath. Upon completion of the addition, the reaction mixture was stirred at 0° until the reaction was completed. Dichloromethane and trifluoroacetic acid were evaporated. The above residue was dissolved in 20 mL methanol, in order added with triethylamine (0.922 mL, 6.6 mmol), dimethyl-carbamic acid 5-oxo-octahydro-pentalen-2-yl ester 4b (460 mg, 2.2 mmol) and sodium triacetoxy borohydride (1.86 g, 8.8 mmol). The reaction mixture was stirred at room temperature, concentrated under reduced pressure, added with 20 mL saturated sodium carbonate solution, extracted with dichloromethane (60 mL×3). The combined organic phase was washed with 15 mL saturated brine, the dichloromethane was dried over anhydrous sodium sulfate, filtered and concentrated under reduced pressure. The residue was purified by silica gel column chromatography to give the title compound 5-[2-(2-cyano-pyrrolidin-1-yl)-2-oxo-ethylamino]-octahydro-pentalen-2-yl dimethyl-carbamate 4c (400 mg, yield 52%) as a white powder.

MS m/z (ESI): 349.4 [M+1].

¹H NMR (400 MHz, CDCl₃) δ (ppm) 5.06 (m, 1H), 4.79 (m, 1H), 4.01-3.37 (m, 4H), 2.89 (s, 6H), 2.42 (m, 2H), 2.36-2.05 (m, 9H), 1.62 (m, 2H), 1.54-1.26 (m, 2H).

### Preparation of 5-[2-(2-cyano-pyrrolidin-1-yl)-2-oxo-ethylamino]-octahydro-pentalen-2-yl dimethyl-carbamate Hydrochloride 4

5-[2-(2-Cyano-pyrrolidin-1-yl)-2-oxo-ethylamino]-octahydro-pentalen-2-yl dimethyl-carbamate 4c (230 mg, 0.661 mmol) was dispersed in 15 mL ether, then a solution of 0.5N hydrochloric acid in 4 mL ether was added under stirring in an ice-water bath. The resulting solid was centrifuged to give the title compound 5-[2-(2-cyano-pyrrolidin-1-yl)-2-oxo-ethylamino]-octahydro-pentalen-2-yl dimethyl-carbamate hydrochloride 4 (200 mg, yield 78.7%) as a white powder.

MS m/z (ESI): 349.2 [M+1].

### Example 5

### Preparation of 5-[2-(2-cyano-pyrrolidin-1-yl)-2-oxo-ethylamino]-octahydro-pentalen-2-yl Acetate Hydrochloride 5

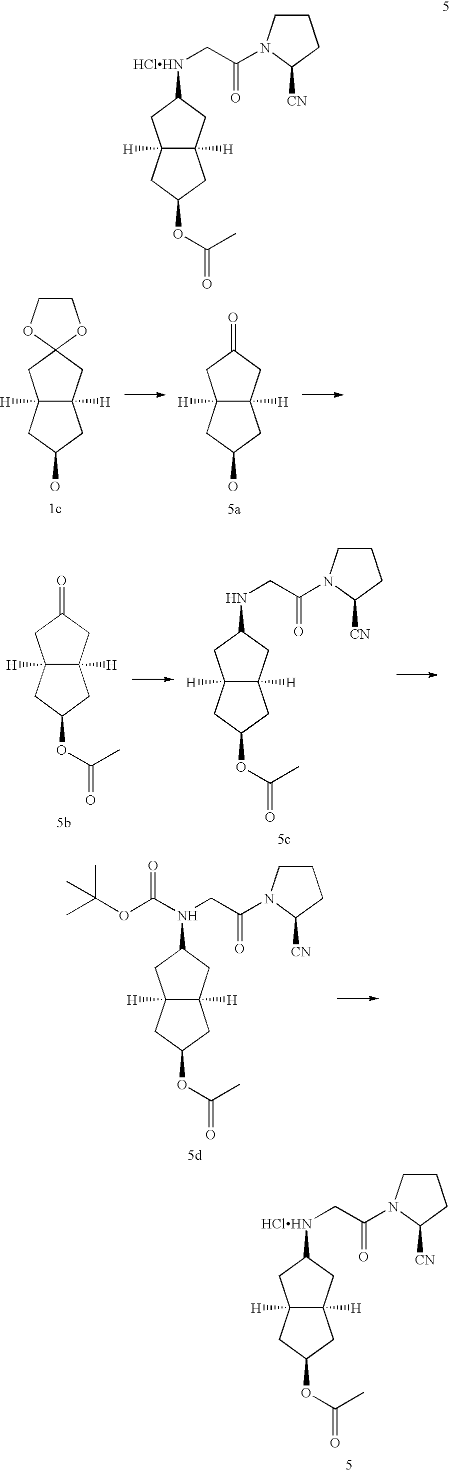

### Preparation of 5-hydroxy-hexahydro-pentalen-2-one 5a

3-Hydroxy-7,7-(ethylidene acetal)bicyclo[3.3.0]octane 1c (2.0 g, 10.87 mmol) was dissolved in the mixture of 60 mL ethyl acetate and 20 mL water, then ethanedioic acid (2.739 g, 21.74 mmol) was added. Upon completion of the addition, the reaction mixture was stirred overnight at room temperature until TLC showed the starting material 1c disappeared, and the reaction mixture was extracted with ethyl acetate (60 mL×3). The combined organic phase was washed with 15 mL saturated brine, the ethyl acetate phase was dried over anhydrous sodium sulfate, filtered and concentrated under reduced pressure. The residue was purified by silica gel column chromatography to give the title compound 5-hydroxy-hexahydro-pentalen-2-one 5a (1.12 g, yield 75%) as a light yellow oil.

MS m/z (ESI): 141.3 [M+1].

### Preparation of 5-oxo-octahydro-pentalen-2-yl Acetate 5b

In a dry three-neck flask, 5-hydroxy-hexahydro-pentalen-2-one 5a (0.7 mg, 5 mmol) was dissolved in pyridine (15 mL), then 4-dimethylamino pyridine (0.18 g, 1.5 mmol) was added, acetic anhydride (0.94 mL, 10 mL) was added dropwise under stirring. Upon completion of the addition, the reaction mixture was heated to reflux overnight. Pyridine was evaporated after the reaction was completed, the reaction mixture was extracted with ethyl acetate (50 mL×3). The combined organic phase was washed with 15 mL saturated brine, the ethyl acetate phase was dried over anhydrous sodium sulfate, filtered and concentrated under reduced pressure. The residue was purified by silica gel column chromatography to give the title compound 5-oxo-octahydro-pentalen-2-yl acetate 5b (480 mg, yield 53.4%) as a light yellow oil.

MS m/z (ESI): 183.6 [M+1].

### Preparation of 5-[2-(2-cyano-pyrrolidin-1-yl)-2-oxo-ethylamino]-octahydro-pentalen-2-yl Acetate 5c

[2-(2-Cyano-pyrrolidin-1-yl)-2-oxo-ethyl]-carbamic acid tert-butyl ester 1f (1.01 mg, 4.0 mmol) was dissolved in 20 mL dichloromethane and trifluoroacetic acid (9.2 mL, 120 mmol) in an ice-water bath. Upon completion of the addition, the reaction mixture was stirred at 0° until the reaction was completed. Dichloromethane and trifluoroacetic acid were evaporated. The above residue was dissolved in 20 mL methanol and in order added with triethylamine (1.12 mL, 8.0 mmol), acetic acid 5-oxo-octahydro-pentalen-2-yl ester 5b (486 mg, 2.67 mmol) and sodium triacetoxy borohydride (1.97 g, 9.34 mmol). The reaction mixture was stirred at room temperature, concentrated and added with 20 mL saturated sodium carbonate solution, extracted with dichloromethane (80 mL×3). The combined organic phase was washed with 15 mL saturated brine, the dichloromethane phase was dried over anhydrous sodium sulfate, filtered and concentrated under reduced pressure. The residue was purified by silica gel column chromatography to give the title compound 5-[2-(2-cyano-pyrrolidin-1-yl)-2-oxo-ethylamino]-octahydro-pentalen-2-yl acetate 5c (950 mg) as a white powder.

MS m/z (ESI): 320.3 [M+1].

### Preparation of 5-{tert-butoxycarbonyl-[2-(2-cyano-pyrrolidin-1-yl)-2-oxo-ethyl]-amino}-octahydro-pentalen-2-yl Acetate 5d

5-[2-(2-Cyano-pyrrolidin-1-yl)-2-oxo-ethylamino]-octahydro-pentalen-2-yl acetate 5c (crude product 950 mg) was dissolved in dichloromethane (30 mL), potassium carbonate (1.38 g, 10 mmol), di-tert-butyl dicarbonate (1.08 g, 5 mmol) was added in order in an ice-water bath. Upon completion of the addition, the reaction mixture was stirred until TLC showed the starting material 5c disappeared and added with water to stop the reaction, the reaction mixture was extracted with dichloromethane (80 mL×3). The combined organic phase was washed with 15 mL saturated brine, the dichloromethane was dried over anhydrous sodium sulfate, filtered and concentrated under reduced pressure. The residue was purified by silica gel column chromatography to give the title compound 5-{tert-butoxycarbonyl-[2-(2-cyano-pyrrolidin-1-yl)-2-oxo-ethyl]-amino}-octahydro-pentalen-2-yl acetate 5d (350 mg, yield 36%) as a white powder.

MS m/z (ESI): 320.4 [M+1].

¹H NMR (400 MHz, CDCl₃) δ (ppm) 5.16 (m, 1H), 4.79 (m, 1H), 3.98-3.37 (m, 4H), 2.45 (m, 2H), 2.36-1.94 (m, 12H), 1.61 (m, 2H), 1.45 (s, 9H), 1.54-1.24 (m, 2H).

### Preparation of 5-[2-(2-cyano-pyrrolidin-1-yl)-2-oxo-ethylamino]-octahydro-pentalen-2-yl Acetate Hydrochloride 5

5-{Tert-butoxycarbonyl-[2-(2-cyano-pyrrolidin-1-yl)-2-oxo-ethyl]-amino}-octahydro-pentalen-2-yl acetate 5d (350 mg, 0.835 mmol) was dispersed in 20 mL ether, a solution of 0.5N hydrochloric acid was added in 4 mL ether in an ice-water bath. After TLC showed the starting material 5d disappeared, the resulting solid was separated by preparative chromatography to give the title compound 5-[2-(2-cyano-pyrrolidin-1-yl)-2-oxo-ethylamino]-octahydro-pentalen-2-yl acetate hydrochloride 5 (140 mg, yield 47%) as a white powder.

MS m/z (ESI): 320.3 [M+1].

### Example 6

### Preparation of 5-[2-(2-cyano-pyrrolidin-1-yl)-2-oxo-ethylamino]-octahydro-pentalen-2-yl phenyl-carbamate Hydrochloride 6

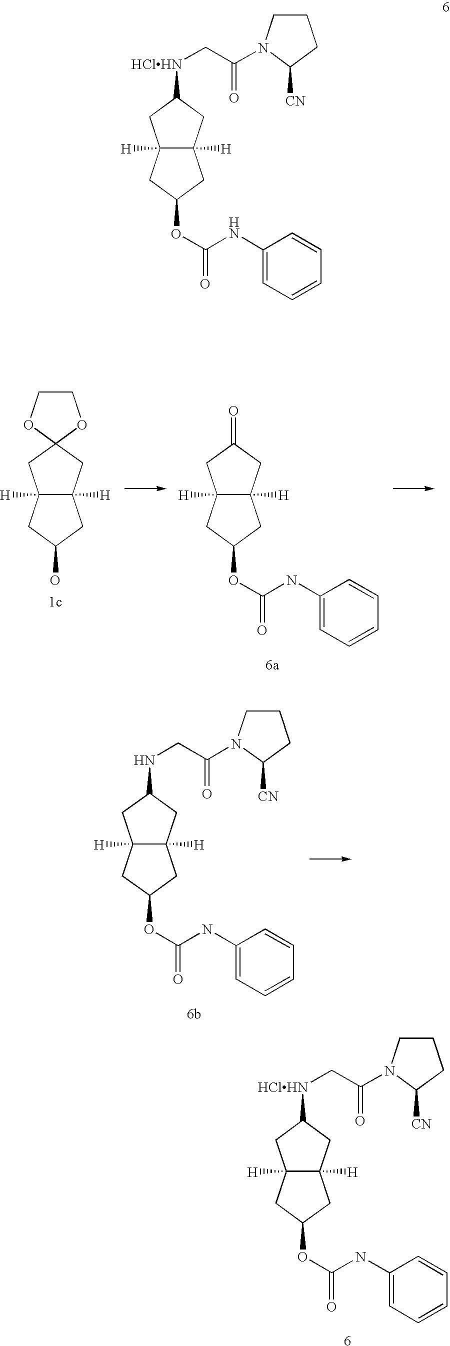

### Preparation of 5-oxo-octahydro-pentalen-2-yl phenyl-carbamate 6a

Tricarbonyl chloride (2.97 g, 0.1 mol) was dissolved in 20 mL toluene, a solution of amidobenzene (2.73 mL, 0.3 mol) in 10 mL toluene was added dropwise under stirring in an ice-water bath. Upon completion of the addition, the reaction mixture was stirred for 0.5 hour at room temperature, for another 2 hours at 50°, then heated to reflux for 2 hours and cooled to room temperature. The reaction mixture was filtered to remove insoluble substance.

3-Hydroxy-7,7-(ethylidene acetal)bicyclo[3.3.0] octane 1c (1.0 g, 5.4 mmol) was dissolved in 20 mL ethylene chloride in an ice-water bath, then the above isocyanate and trimethychlorosilicane (70 ul, 0.55 mmol) were added, then naturally raised to room temperature. The reaction mixture was stirred overnight at room temperature, added with water to stop the reaction, extracted with dichloromethane (30 mL×3). The combined organic phase was washed with 30 mL saturated brine, the dichloromethane phase was dried over anhydrous sodium sulfate, filtered and concentrated under reduced pressure.

The concentration was added with 20 mL ethyl acetate and 5 mL 2N hydrochloric acid, stirred for 1 hour at 35Ξ and extracted with ethyl acetate (30 mL×3). The combined organic phase was washed with 30 mL saturated brine, the ethyl acetate was dried over anhydrous sodium sulfate, filtered and concentrated under reduced pressure. The residue was purified by silica gel column chromatography to give the title compound 5-oxo-octahydro-pentalen-2-yl phenyl-carbamate 6a (1.0 g, yield 75%) as a white powder.

MS m/z (ESI): 260.4 [M+1].

### Preparation of 5-[2-(2-cyano-pyrrolidin-1-yl)-2-oxo-ethylamino]-octahydro-pentalen-2-yl phenyl-carbamate 6b

[2-(2-Cyano-pyrrolidin-1-yl)-2-oxo-ethyl]-carbamic acid tert-butyl ester 1f (750 mg, 2.96 mmol) was dissolved in 20 mL dichloromethane, then trifluoroacetic acid (6.8 mL, 90 mmol) was added under stirring in an ice-water bath. Upon completion of the addition, the reaction mixture was stirred at 0° until the reaction was completed. Dichloromethane and trifluoroacetic acid were evaporated. The above residue was dissolved in 20 mL methanol, in order added with triethylamine (1.03 mL, 7.1 mmol), 5-oxo-octahydro-pentalen-2-yl phenyl-carbamate 6a (640 mg, 2.47 mmol) and sodium triacetoxy borohydride (2.1 g, 9.9 mmol). The reaction mixture was stirred at room temperature, concentrated and added with 20 mL saturated sodium carbonate solution, extracted with dichloromethane (80 mL×3). The combined organic phase was washed with 15 mL saturated brine, the dichloromethane was dried over anhydrous sodium sulfate, filtered and concentrated under reduced pressure. The residue was purified by silica gel column chromatography to give the title compound 5-[2-(2-cyano-pyrrolidin-1-yl)-2-oxo-ethylamino]-octahydro-pentalen-2-yl phenyl-carbamate 6b (300 mg, yield 30.6%) as a white powder.

MS m/z (ESI): 397.6 [M+1].

¹H NMR (400 MHz, CDCl₃) δ (ppm) 7.55 (m, 1H), 7.27 (m, 3H), 7.01 (m, 1H), 5.32 (m, 1H), 4.72 (m, 1H), 4.32-3.93 (m, 2H), 3.65 (m, 2H), 2.55 (m, 2H), 2.40-1.95 (m, 7H), 1.94-1.60 (m, 6H).

### Preparation of 5-[2-(2-cyano-pyrrolidin-1-yl)-2-oxo-ethylamino]-octahydro-pentalen-2-yl phenyl-carbamate Hydrochloride 6

5-[2-(2-Cyano-pyrrolidin-1-yl)-2-oxo-ethylamino]-octahydro-pentalen-2-yl phenyl-carbamate 6b (300 mg, 0.758 mmol) was dispersed in 15 mL ether, then a solution of 0.5N hydrochloric acid in 2 mL ether was added in an ice-water bath. The resulting solid was centrifuged to give the title compound 5-[2-(2-cyano-pyrrolidin-1-yl)-2-oxo-ethylamino]-octahydro-pentalen-2-yl phenyl-carbamate hydrochloride 6 (200 mg, yield 61%) as a white powder.

MS m/z (ESI): 397.3 [M+1].

### Example 7

### Preparation of 5-[2-(2-cyano-pyrrolidin-1-yl)-2-oxo-ethylamino]-octahydro-pentalen-2-yl isopropyl-carbamate Hydrochloride 7

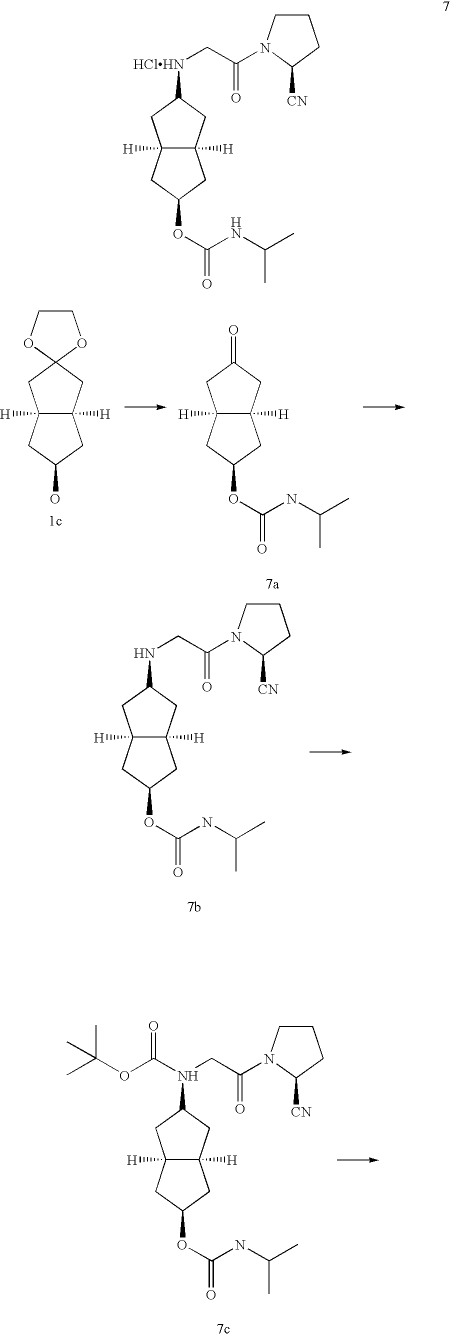

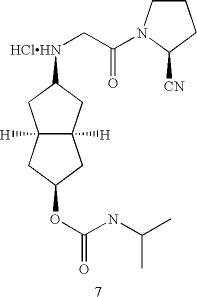

### Preparation of 5-oxo-octahydro-pentalen-2-yl isopropyl-carbamate 7a

Tricarbonyl chloride (2.97 g, 0.1 mol) was dissolved in 20 mL toluene, then a solution of isopropylamine (2.6 mL, 30 mmol) in 10 mL toluene was added dropwise in an ice-water bath. Upon completion of the addition, the reaction mixture was stirred for 0.5 hour at room temperature, for another 2 hours at 500, then heated to reflux for 2 hours and cooled to room temperature. The reaction mixture was filtered to remove insoluble substance.

3-Hydroxy-7,7-(ethylidene acetal)bicyclo[3.3.0] octane 1c (1.0 g, 5.4 mmol) was dissolved in ethylene chloride (20 mL) in an ice-water bath, the above isocyanate and trimethychlorosilicane (70 ul, 0.55 mmol) were added, then naturally raised to room temperature. Upon completion of the addition, the reaction mixture was stirred overnight at room temperature and added with water to stop the reaction, the reaction mixture was extracted with dichloromethane (80 mL×3). The combined organic phase was washed with 15 mL saturated brine, the dichloromethane was dried over anhydrous sodium sulfate, filtered and concentrated under reduced pressure.

The concentration was added with 20 mL ethyl acetate and 5 mL 2N hydrochloric acid, stirred for 1 hour at 35° and extracted with ethyl acetate (80 mL×3). The combined organic phase was washed with 15 mL saturated brine, the ethyl phase was dried over anhydrous sodium sulfate, filtered and concentrated under reduced pressure. The residue was purified by silica gel column chromatography to give the title compound 5-oxo-octahydro-pentalen-2-yl isopropyl-carbamate 7a (1.01 g, yield 81.8%) as a white powder.

MS m/z (ESI): 226.2 [M+1].

### Preparation of 5-[2-(2-cyano-pyrrolidin-1-yl)-2-oxo-ethylamino]-octahydro-pentalen-2-yl isopropyl-carbamate 7b

[2-(2-Cyano-pyrrolidin-1-yl)-2-oxo-ethyl]-carbamic acid tert-butyl ester 1f (843 mg, 3.33 mmol) was dissolved in 30 mL dichloromethane, then trifluoroacetic acid (7.66 mL, 100 mmol) was added in an ice-water bath. Upon completion of the addition, the reaction mixture was stirred at 0° until the reaction was completed. Dichloromethane and trifluoroacetic acid were evaporated. The above residue was dissolved in 20 mL methanol and in order added with triethylamine (1.3 mL, 9.6 mmol), isopropyl-carbamic acid 5-oxo-octahydro-pentalen-2-yl ester 7a (500 mg, 2.22 mmol) and sodium triacetoxy borohydride (2.2 g, 10 mmol). The reaction mixture was stirred at room temperature, concentrated under reduced pressure and added with 20 mL saturated sodium carbonate solution, then extracted with dichloromethane (60 mL×3). The combined organic phase was washed with 15 mL saturated brine, the dichloromethane phase was dried over anhydrous sodium sulfate, filtered and concentrated under reduced pressure to give the title compound 5-[2-(2-cyano-pyrrolidin-1-yl)-2-oxo-ethylamino]-octahydro-pentalen-2-yl isopropyl-carbamate 7b (1.9 g) as a white powder, the crude product was directly used in the further reaction.

MS m/z (ESI): 363.5 [M+1].

### Preparation of 5-{tert-butoxycarbonyl-[2-(2-cyano-pyrrolidin-1-yl)-2-oxo-ethyl]-amino}-octahydro-pentalen-2-yl isopropyl-carbamate 7c

5-[2-(2-Cyano-pyrrolidin-1-yl)-2-oxo-ethylamino]-octahydro-pentalen-2-yl isopropyl-carbamate 7b (1.9 g, 2.22 mmol) was dissolved in dichloromethane (30 mL), then potassium carbonate (1.53 g, 11.1 mmol), di-tert-butyl dicarbonate (1.21 g, 5.35 mmol) were added in order in an ice-water bath. Upon completion of the addition, the reaction mixture was stirred at room temperature until the starting material 7b disappeared and added with water to stop the reaction, the reaction mixture was extracted with dichloromethane (60 mL×3). The combined organic phase was washed with 15 mL saturated brine, the dichloromethane was dried over anhydrous sodium sulfate, filtered and concentrated under reduced pressure. The residue was purified by silica gel column chromatography to give the title compound 5-{tert-butoxycarbonyl-[2-(2-cyano-pyrrolidin-1-yl)-2-oxo-ethyl]-amino}-octahydro-pentalen-2-yl isopropyl-carbamate 7c (630 mg, yield 61.2%) as a white powder.

MS m/z (ESI): 463.3 [M+1].

¹H NMR (400 MHz, CDCl₃) δ (ppm) 5.15 (m, 1H), 4.76 (m, 1H), 4.01 (m, 2H), 3.81 (m, 2H), 3.65 (m, 2H), 2.45 (m, 2H), 2.38-2.00 (m, 6H), 1.98-1.60 (m, 6H), 1.46 (s, 9H), 1.18 (m, 6H).

### Preparation of 5-[2-(2-cyano-pyrrolidin-1-yl)-2-oxo-ethylamino]-octahydro-pentalen-2-yl isopropyl-carbamate Hydrochloride 7

In a dry three-neck flask, 30 ml dichloromethane was added, 5-{tert-butoxycarbonyl-[2-(2-cyano-pyrrolidin-1-yl)-2-oxo-ethyl]-amino}-octahydro-pentalen-2-yl isopropyl-carbamate 7c (360 mg, 0.779 mmol) was dispersed in 15 mL ether, then a solution of 0.5N hydrochloric acid in 3 mL ether was added in an ice-water bath. After the staring material 7c disappeared, the solvent was evaporated under reduce pressure. The residue was separated by column chromatography to give the title compound 5-[2-(2-cyano-pyrrolidin-1-yl)-2-oxo-ethylamino]-octahydro-pentalen-2-yl isopropyl-carbamate hydrochloride 7 (150 mg, yield 66%) as a white powder.

MS m/z (ESI): 363.1 [M+1].

### Example 8

### Preparation of 5-[2-(2-cyano-pyrrolidin-1-yl)-2-oxo-ethylamino]-octahydro-pentalen-2-yl tert-butyl-carbamate Hydrochloride 8

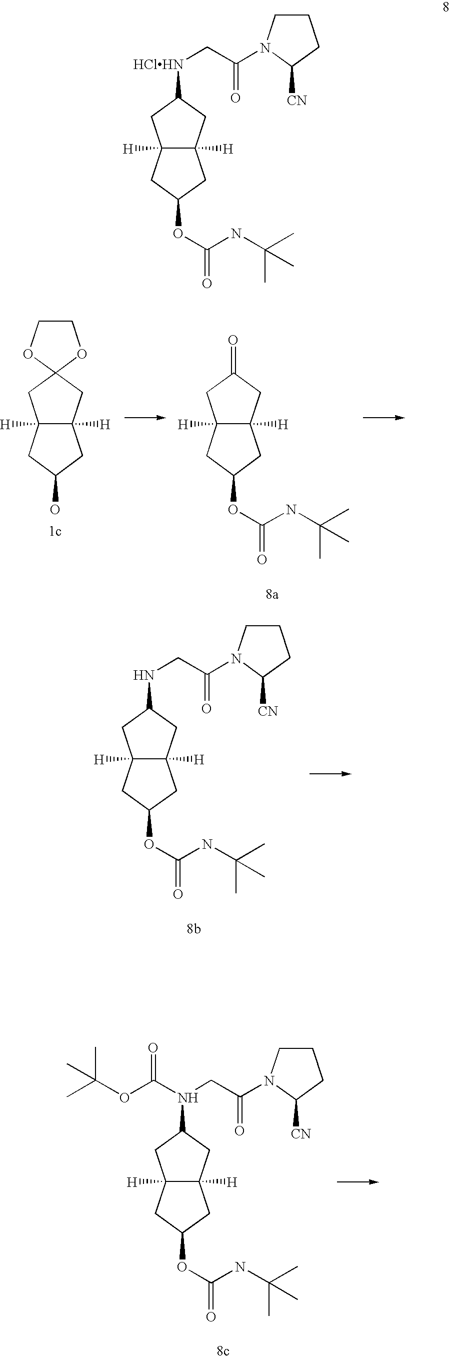

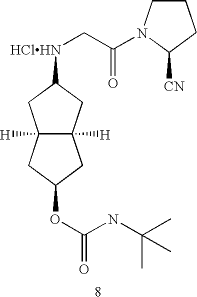

### Preparation of 5-oxo-octahydro-pentalen-2-yl tert-butyl-carbamate 8a

Tricarbonyl chloride (2.97 g, 0.1 mol) was dissolved in toluene (20 mL), then a solution of tert-butylamine (3.16 mL, 30 mmol) in toluene (10 mL) was added dropwise in an ice-water bath. Upon completion of the addition, the reaction mixture was stirred for 0.5 hour at room temperature, for another 2 hours at 50°, then heated to reflux for 2 hours and cooled to room temperature. The reaction mixture was filtered to remove insoluble substance.

3-Hydroxy-7,7-(ethylidene acetal)bicyclo[3.3.0] octane 1c (1.0 g, 5.4 mmol) was dissolved in ethylene chloride (20 mL), and the above isocyanate and trimethychlorosilicane (70 ul, 0.55 mmol) was added in an ice-water bath, then naturally raised to room temperature. Upon completion of the addition, the reaction mixture was stirred overnight at room temperature and added with water to stop the reaction, the reaction mixture was extracted with dichloromethane (80 mL×3). The combined organic phase was washed with 15 mL saturated brine, the dichloromethane was dried over anhydrous sodium sulfate, filtered and concentrated under reduced pressure.

The concentration was added with 10 mL ethyl acetate and 5 mL 2N hydrochloric acid, stirred for 1 hour at 35° and extracted with ethyl acetate (60 mL×3). The combined organic phase was washed with 15 mL saturated brine, the ethyl acetate was dried over anhydrous sodium sulfate, filtered and concentrated under reduced pressure. The residue was purified by silica gel column chromatography to give the title compound 5-oxo-octahydro-pentalen-2-yl tert-butyl-carbamate 8a (1.03 g, yield 79.4%) as a white powder.

MS m/z (ESI): 240.2 [M+1].

### Preparation of 5-[2-(2-cyano-pyrrolidin-1-yl)-2-oxo-ethylamino]-octahydro-pentalen-2-yl tert-butyl-carbamate 8b

[2-(2-Cyano-pyrrolidin-1-yl)-2-oxo-ethyl]-carbamic acid tert-butyl ester 1f (794 mg, 3.318 mmol) was dissolved in 30 mL dichloromethane, then trifluoroacetic acid (7.2 mL, 94.18 mmol) was added in an ice-water bath. Upon completion of the addition, the reaction mixture was stirred at 0° until the reaction was completed. Dichloromethane and trifluoroacetic acid were evaporated. The above residue was dissolved in 20 mL methanol and in order added with triethylamine (0.87 mL, 6.276 mmol), 5-oxo-octahydro-pentalen-2-yl tert-Butyl-carbamate 8a (500 mg, 2.092 mmol) and sodium triacetoxy borohydride (1.744 g, 8.368 mmol). The reaction mixture was stirred at room temperature, concentrated under the reduced pressure and added with 20 mL saturated sodium carbonate solution, extracted with dichloromethane (80 mL×3). The combined organic phase was washed with 15 mL saturated brine, the dichloromethane phase was dried over anhydrous sodium sulfate, filtered and concentrated under reduced pressure to give the title compound 5-[2-(2-cyano-pyrrolidin-1-yl)-2-oxo-ethylamino]-octahydro-pentalen-2-yl tert-butyl-carbamate 8b (1.5 g) as a white powder, the crude product was directly used in the further reaction.

MS m/z (ESI): 377.3 [M+1].

### Preparation of 5-{tert-butoxycarbonyl-[2-(2-cyano-pyrrolidin-1-yl)-2-oxo-ethyl]-amino}-octahydro-pentalen-2-yl tert-butyl-carbamate 8c

5-[2-(2-Cyano-pyrrolidin-1-yl)-2-oxo-ethylamino]-octahydro-pentalen-2-yl tert-butyl-carbamate 8b (1.5 g, 2.09 mmol) was dissolved in 30 mL dichloromethane, potassium carbonate (1.44 g, 10.46 mmol), di-tert-butyl dicarbonate (1.14 g, 5.23 mmol) were added in order in an ice-water bath. Upon completion of the addition, the reaction mixture was stirred until the starting material 8b disappeared, then added with water to stop the reaction, the reaction mixture was extracted with dichloromethane (60 mL×3). The combined organic phase was washed with 15 mL saturated brine, the dichloromethane was dried over anhydrous sodium sulfate, filtered and concentrated under reduced pressure. The residue was purified by silica gel column chromatography to give the title compound 5-{tert-butoxycarbonyl-[2-(2-cyano-pyrrolidin-1-yl)-2-oxo-ethyl]-amino}-octahydro-pentalen-2-yl tert-butyl-carbamate 8c (280 mg, yield 28.1%) as a white powder.

MS m/z (ESI): 477.1 [M+1].

### Preparation of 5-[2-(2-cyano-pyrrolidin-1-yl)-2-oxo-ethylamino]-octahydro-pentalen-2-yl tert-butyl-carbamate Hydrochloride 8

In a dry three-neck flask, 30 ml dichloromethane was added, 5-{tert-butoxycarbonyl-[2-(2-cyano-pyrrolidin-1-yl)-2-oxo-ethyl]-amino}-octahydro-pentalen-2-yl tert-butyl-carbamate 8c (210 mg, 0.441 mmol) was dispersed in 15 mL ether, then a solution of 0.5N hydrochloric acid in 3 mL ether was added in an ice-water bath. After the staring material 8c disappeared, the solvent was evaporated under reduce pressure. The residue was separated by column chromatography to give the title compound 5-[2-(2-cyano-pyrrolidin-1-yl)-2-oxo-ethylamino]-octahydro-pentalen-2-yl tert-butyl-carbamate hydrochloride 8 (100 mg, yield 55%) as a white powder.

MS m/z (ESI): 377.5 [M+1].

¹H NMR (400 MHz, CDCl₃) δ (ppm) 5.11 (m, 1H), 4.76 (m, 1H), 3.64 (m, 2H), 2.44 (m, 2H), 2.33-2.09 (m, 8H), 2.06 (m, 2H), 1.74-1.64 (m, 5H), 1.47 (s, 9H), 1.33 (m, 9H).

### Example 9

### Preparation of 1-[2-(5-ethyl-5-hydroxy-octahydro-pentalen-2-ylamino)-1-hydroxy-ethyl]-pyrrolidine-2-carbonitrile Hydrochloride 9

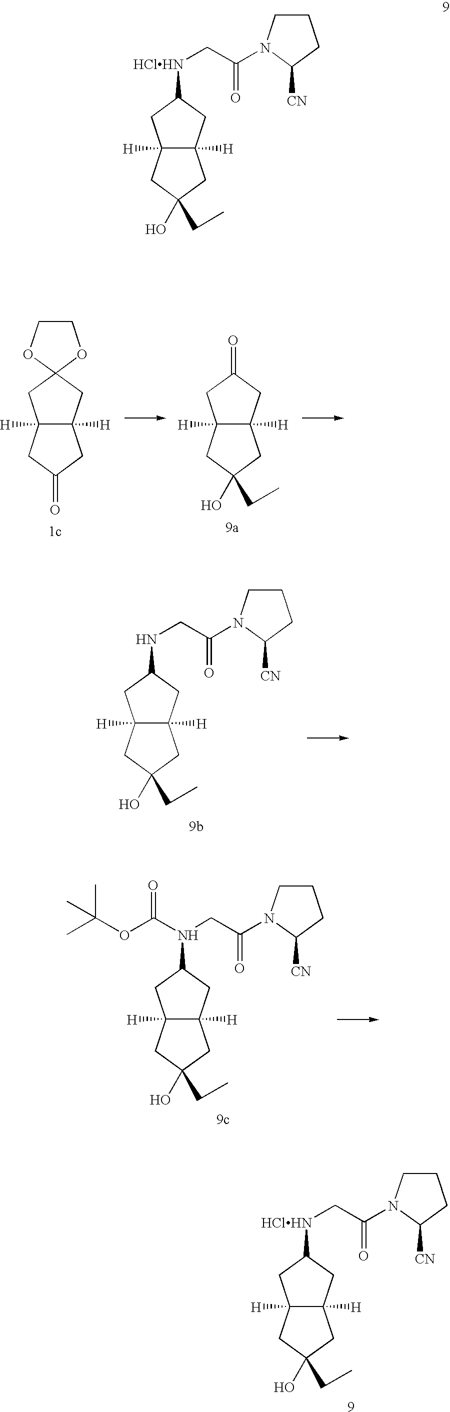

### Preparation of 5-ethyl-5-hydroxy-hexahydro-pentalen-2-one 9a

Magnesium (243 mg, 10 mmol) was added to 20 mL ether and lightly heated. The mixture was added with catalytic amount iodine and dropwise ethyl bromide (0.75 mL, 10 mmol) while maintaining the reaction system lightly boiled for 1 hour. The reaction mixture was dropwise added with a solution of 7,7-(ethylidene acetal)bicyclo[3.3.0] octane-3-one 1b (0.8 g, 4.4 mmol) in 5 mL ether and then stirred for 2 hours, added with 10 mL 2N hydrochloric acid to stop the reaction for another 2 hours stirring, then the reaction mixture was extracted with ethyl acetate (30 mL×3). The combined organic phase was washed with 30 mL saturated brine, the ethyl acetate phase was dried over anhydrous sodium sulfate, filtered and concentrated under reduced pressure. The residue was purified by silica gel column chromatography to give the title compound 5-ethyl-5-hydroxy-hexahydro-pentalen-2-one 9a (420 mg, yield 57%) as a light yellow oil.

MS m/z (ESI): 169.2 [M+1].

### Preparation of 1-[2-(5-ethyl-5-hydroxy-octahydro-pentalen-2-ylamino)-acetyl]-pyrrolidine-2-carbonitrile 9b

[2-(2-Cyano-pyrrolidin-1-yl)-2-oxo-ethyl]-carbamic acid tert-butyl ester 1f (949 mg, 3.75 mmol) was dissolved in 30 mL dichloromethane, trifluoroacetic acid (8.62 mL, 112.5 mmol) was added in an ice-water bath. Upon completion of the addition, the reaction mixture was stirred at 0° until the reaction was completed. Dichloromethane and trifluoroacetic acid were evaporated. The above residue was dissolved in 20 mL methanol and in order added with triethylamine (1.04 mL, 7.5 mmol), 5-ethyl-5-hydroxy-hexahydro-pentalen-2-one 9a (420 mg, 2.5 mmol) and sodium triacetoxy borohydride (2.385 g, 11.25 mmol). The reaction mixture was stirred o at room temperature, concentrated under reduced pressure and added with 20 mL saturated sodium carbonate solution, then extracted with dichloromethane (80 mL×3). The combined organic phase was washed with 15 mL saturated brine, the dichloromethane phase was dried over anhydrous sodium sulfate, filtered and concentrated under reduced pressure to give the title compound 1-[2-(5-ethyl-5-hydroxy-octahydro-pentalen-2-ylamino)-acetyl]-pyrrolidine-2-carbonitrile 9b (700 mg) as a white powder, the crude product was directly used in the further reaction.

MS m/z (ESI): 306.4 [M+1].

### Preparation of tert-butyl-[2-(2-cyano-pyrrolidin-1-yl)-2-oxo-ethyl]-(5-ethyl-5-hydroxy-octahydro-pentalen-2-yl)-carbamate 9c

1-[2-(5-Ethyl-5-hydroxy-octahydro-pentalen-2-ylamino)-acetyl]-pyrrolidine-2-carbonitrile 9b (700 mg, 2.5 mmol) was dissolved in 30 mL dichloromethane, then potassium carbonate (1.09 g, 6.25 mmol), di-tert-butyl dicarbonate (1.38 g, 12.5 mmol) were added in order in an ice-water bath. Upon completion of the addition, the reaction mixture was stirred at room temperature until the starting material 9b disappeared, then added with water to stop the reaction, the reaction mixture was extracted with dichloromethane (80 mL×3). The combined organic phase was washed with 15 mL saturated brine, the dichloromethane phase was dried over anhydrous sodium sulfate, filtered and concentrated under reduced pressure. The residue was purified by silica gel column chromatography to give the title compound tert-butyl-[2-(2-cyano-pyrrolidin-1-yl)-2-oxo-ethyl]-(5-ethyl-5-hydroxy-octahydro-pentalen-2-yl)-carbamate 9c (355 mg, yield 35%) as a white powder.

MS m/z (ESI): 406.2 [M+1].

¹H NMR (400 MHz, CDCl₃) δ (ppm) 4.78 (m, 1H), 3.94-3.89 (m, 3H), 2.48 (m, 2H), 2.34-2.08 (m, 6H), 1.75 (m, 2H), 1.68-1.39 (m, 17H), 0.93 (m, 3H).

### Preparation of 1-[2-(5-ethyl-5-hydroxy-octahydro-pentalen-2-ylamino)-1-hydroxy-ethyl]-pyrrolidine-2-carbonitrilehydrochloride 9

In a dry three-neck flask, 10 ml dichloromethane was added, tert-butyl-[2-(2-cyano-pyrrolidin-1-yl)-2-oxo-ethyl]-(5-ethyl-5-hydroxy-octahydro-pentalen-2-yl)-carbamate 9c (200 mg, 0.4938 mmol) was dispersed in 15 mL ether, then a solution of 0.5N hydrochloric acid in 3 mL ether was added in an ice-water bath. After the staring material 9c disappeared, the solvent was evaporated under reduce pressure. The residue was separated by column chromatography to give the title compound 1-[2-(5-ethyl-5-hydroxy-octahydro-pentalen-2-ylamino)-1-hydroxy-ethyl]-pyrrolidine-2-carbonitrilehydrochloride 9 (100 mg, yield 59.5%) as a white powder.

MS m/z (ESI): 306.5 [M+1].

### Example 10

### Preparation of 1-[2-(5-butyl-5-hydroxy-octahydro-pentalen-2-ylamino)-acetyl]-pyrrolidine-2-carbonitrile Hydrochloride 10

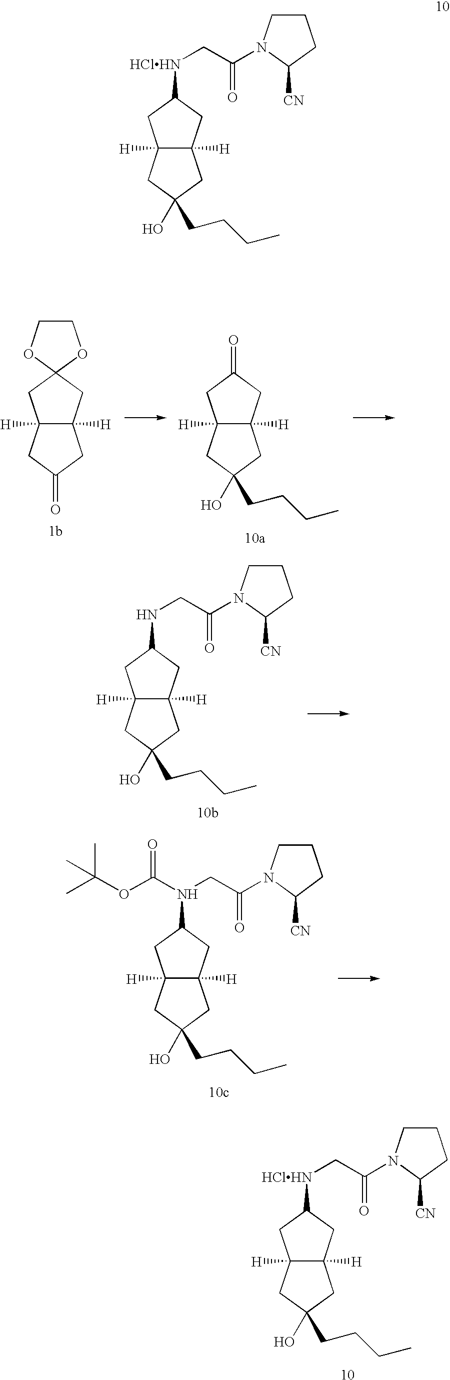

### 5-butyl-5-hydroxy-tetrahydro-pentalen-2-one 10a

In a dry three-neck flask, butyl-magnesium chloride (2N in tetrahydrofuran, 4.8 mL) was dissolved in tetrahydrofuran (15 mL), then a solution of 7,7-(ethylidene acetal)bicyclo[3.3.0] octane-3-one 1b (1.46 g, 8 mmol) in 10 mL tetrahydrofuran was added dropwise in an ice-water bath. Upon completion of the addition, the reaction mixture was stirred for 2 hours, then added with 2 mL 2N hydrochloric acid to stop the reaction for another 2 hours stirring, and extracted with ethyl acetate (80 mL×3). The combined organic phase was washed with saturated brine (15 mL×1), the ethyl acetate phase was dried over anhydrous sodium sulfate, filtered and concentrated under reduced pressure. The residue was purified by silica gel column chromatography to give the title compound 5-butyl-5-hydroxy-tetrahydro-pentalen-2-one 10a (620 mg, yield 35%) as a light yellow oil.

MS m/z (ESI): 197.2 [M+1].

### Preparation of 1-(2-(5-butyl-5-hydroxy-octahydro-pentalen-2-ylamino)-acetyl)-pyrrolidine-2-carbonitrile 10b

[2-(2-Cyano-pyrrolidin-1-yl)-2-oxo-ethyl]-carbamic acid tert-butyl ester 1f (1.20 g, 4.7 mmol) was dissolved in 20 mL dichloromethane, then trifluoroacetic acid (10.8 mL, 141 mmol) was added in an ice-water bath. Upon completion of the addition, the reaction mixture was stirred at 0° until reaction was completed. Dichloromethane and trifluoroacetic acid were evaporated. The above residue was dissolved in 20 mL methanol and in order added with triethylamine (1.32 mL, 9.5 mmol), 5-butyl-5-hydroxy-hexahydro-pentalen-2-one 10a (620 mg, 3.16 mmol) and sodium triacetoxy borohydride (3.0 g, 14.2 mmol). The reaction mixture was stirred at room temperature, concentrated under reduced pressure, and added with 20 mL saturated sodium carbonate solution, then extracted with dichloromethane (80 mL×3). The combined organic phase was washed with 15 mL saturated brine, the dichloromathine phase was dried over anhydrous sodium sulfate, filtered and concentrated under reduced pressure to give 1-(2-(5-butyl-5-hydroxy-octahydro-pentalen-2-ylamino)-acetyl)-pyrrolidine-2-carbonitrile 10b (1.0 g) as a light yellow oil, the crude product was directly used in the further reaction.

MS m/z (ESI): 334.5 [M+1].

¹H NMR (400 MHz, CD₃OD) δ (ppm) 4.61 (m, 1H), 3.48 (m, 1H), 3.34 (m, 3H), 2.90 (m, 1H), 2.32 (m, 2H), 2.10 (m, 2H), 1.98 (m, 4H), 1.70 (m, 2H), 1.43 (m, 2H), 1.31 (m, 4H), 1.17 (m, 4H), 0.76 (m, 3H).

### Preparation of tert-butyl-(5-Butyl-5-hydroxy-octahydro-pentalen-2-yl)-[2-(2-cyano-pyrrolidin-1-yl)-2-oxo-ethyl]-carbamate 10c

1-[2-(5-Butyl-5-hydroxy-octahydro-pentalen-2-ylamino)-acetyl]-pyrrolidine-2-carbonitrile 10b (900 mg, 2.7 mmol) was dissolved in 20 mL dichloromethane, then potassium carbonate (1.38 g, 10 mmol), di-tert-butyl dicarbonate (1.08 g, 5 mmol) were added in order in an ice-water bath. Upon completion of the addition, the reaction mixture was stirred until the starting material 10b disappeared, then added with water to stop the reaction, the reaction mixture was extracted with dichloromethane (80 mL×3). The combined organic phase was washed with 15 mL saturated brine, the dichloromethane phase was dried over anhydrous sodium sulfate, filtered and concentrated under reduced pressure. The residue was purified by silica gel column chromatography to give the title compound tert-butyl-(5-butyl-5-hydroxy-octahydro-pentalen-2-yl)-[2-(2-cyano-pyrrolidin-1-yl)-2-oxo-ethyl]-carbamate 10c (350 mg, yield 30%) as a white powder.

MS m/z (ESI): 434.3 [M+1].

### Preparation of 1-[2-(5-butyl-5-hydroxy-octahydro-pentalen-2-ylamino)-acetyl]-pyrrolidine-2-carbonitrilehydrochloride 10

In a dry three-neck flask, 10 ml dichloromethane was added, tert-butyl-(5-butyl-5-hydroxy-octahydro-pentalen-2-yl)-[2-(2-cyano-pyrrolidin-1-yl)-2-oxo-ethyl]-carbamate 10c (200 mg, 0.462 mmol) was disperse in 15 mL ether, then a solution of 0.5N hydrochloric acid in 3 mL ether was added in an ice-water bath. After the staring material 10c disappeared, the solvent was evaporated under reduce pressure. The residue was separated by column chromatography to give the title compound 1-[2-(5-butyl-5-hydroxy-octahydro-pentalen-2-ylamino)-acetyl]-pyrrolidine-2-carbonitrilehydrochloride 10 (75 mg, yield 44%) as a white powder.

MS m/z (ESI): 334.2 [M+1].

### Example 11

### Preparation of 1-[2-(5-isopropyl-5-hydroxy-octahydro-pentalen-2-ylamino) acetyl]pyrrolidine-2-carbonitrile Hydrochloride 11

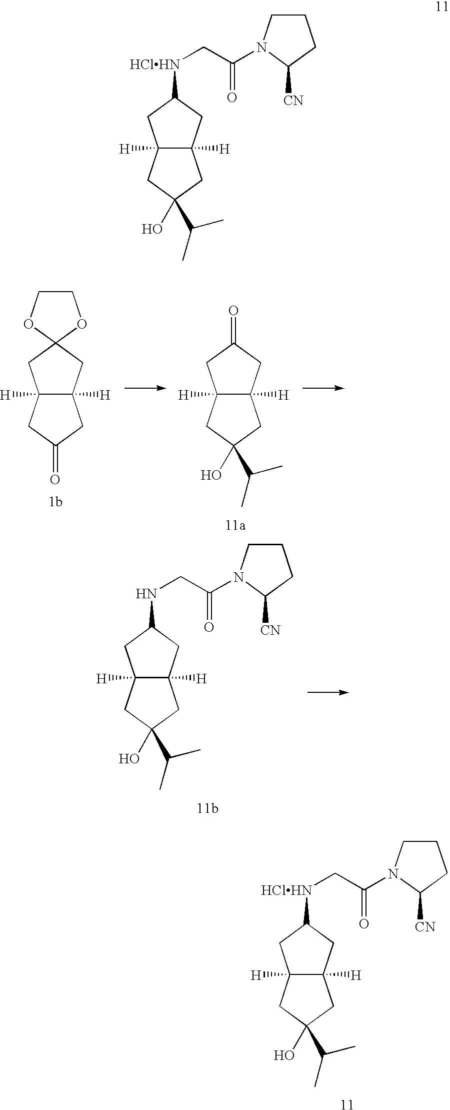

### Preparation of 5-hydroxy-5-isopropyl-hexahydro-pentalen-2-one 11a

Magnesium (4.92 g, 40 mmol) was added to 10 mL ether and lightly heated. The mixture was stirred and added with catalytic amount iodine and dropwise 2-bromo propane (40 mL, 40 mmol) while maintaining the reaction system lightly boiled for 1 hour. The reaction mixture was dropwise added with a solution of 7,7-(ethylidene acetal)bicyclo[3.3.0] octane-3-one 1b (1.82 g, 10 mmol) in 10 mL ether and then stirred for 2 hours, added with 2 mL 2N hydrochloric acid to stop the reaction for another 2 hours stirring and extracted with ethyl acetate (80 mL×3). The combined organic phase was washed with 15 mL saturated brine, the ethyl acetate phase was dried over anhydrous sodium sulfate, filtered and concentrated under reduced pressure. The residue was purified by silica gel column chromatography to give the title compound 5-hydroxy-5-isopropyl-hexahydro-pentalen-2-one 11a (270 mg, yield 15%) as a light yellow oil.

MS m/z (ESI): 282.2 [M+1].

### Preparation of 1-[2-(5-isopropyl-5-hydroxy-octahydro-pentalen-2-ylamino)-acetyl]pyrrolidine-2-carbonitrile 11b

[2-(2-Cyano-pyrrolidin-1-yl)-2-oxo-ethyl]-carbamic acid tert-butyl ester 1f (562 mg, 2.22 mmol) was dissolved in 20 mL dichloromethane, then trifluoroacetic acid (5.12 mL, 66.8 mmol) was added in an ice-water bath. Upon completion of the addition, the reaction mixture was stirred at 0° until reaction was completed. Dichloromethane and trifluoroacetic acid were evaporated. The above residue was dissolved in 20 mL methanol and in order added with triethylamine (0.62 mL, 4.44 mmol), 5-hydroxy-5-isopropyl-hexahydro-pentalen-2-one 11a (270 mg, 1.48 mmol) and sodium triacetoxy borohydride (1.41 g, 6.66 mmol). The reaction mixture was stirred at room temperature, concentrated under reduce pressure and added with 20 mL saturated sodium carbonate solution, then extracted with dichloromethane (80 mL×3). The combined organic phase was washed with 15 mL saturated brine, the dichloromethane was dried over anhydrous sodium sulfate, filtered and concentrated under reduced pressure. The residue was purified by silica gel column chromatography to give the title compound 1-(2-(5-isopropyl-5-hydroxy-octahydro-pentalen-2-ylamino)-acetyl)pyrrolidine-2-carbonitrile 11b (90 mg, yield 20%) as a white powder.

MS m/z (ESI): 320.4 [M+1].

### Preparation of 1-[2-(5-isopropyl-5-hydroxy-octahydro-pentalen-2-ylamino)-acetyl]pyrrolidine-2-carbonitrile Hydrochloride 11

1-[2-(5-Isopropyl-5-hydroxy-octahydro-pentalen-2-ylamino)-acetyl]pyrrolidine-2-carbonitrile 11a (90 mg, 0.282 mmol) was dispersed in 10 mL ether, then a solution of 0.5N hydrochloric acid in 2 mL ether was added in an ice-water bath. The resulting solid was centrifuged to give the title compound 1-(2-(5-isopropyl-5-hydroxy-octahydro-pentalen-2-ylamino)-acetyl)pyrrolidine-2-carbonitrile hydrochloride 11 (80 mg, yield 80%) as a light yellow powder.

MS m/z (ESI): 320.5 [M+1].

### Example 12

### Preparation of 1-{2-[5-(4-fluoro-phenyl)-5-hydroxy-octahydro-pentalen-2-ylamino]-acetyl}-pyrrolidine-2-carbonitrile Hydrochloride 12

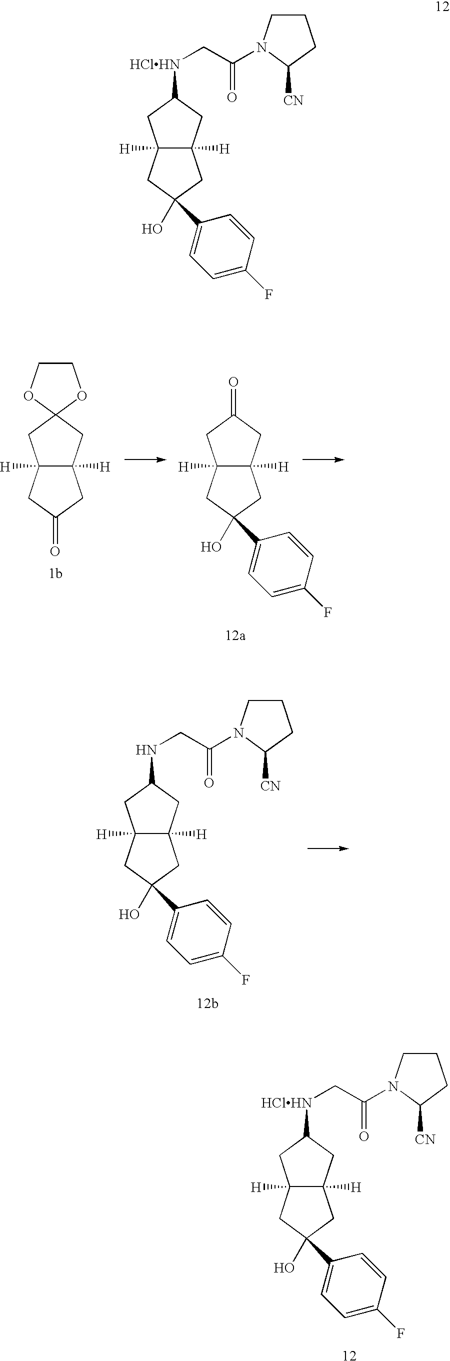

### Preparation of 5-(4-fluoro-phenyl)-5-hydroxy-hexahydro-pentalen-2-one 12a

Magnesium (486 mg, 20 mmol) was added to 20 mL ether and lightly heated. The mixture was stirred and added with catalytic amount iodine and dropwise 1-bromo-4-fluoro-benzene (2.4 mL, 22 mmol) while maintaining the reaction system lightly boiled for 1 hour. The reaction mixture was dropwise added with a solution of 7,7-(ethylidene acetal)bicyclo[3.3.0] octane-3-one 1b (1.19 g, 6.54 mmol) in 10 mL ether and then was stirred for 2 hours, added with 2 mL 2N hydrochloric acid to stop the reaction for another 2 hours stirring and extracted with ethyl acetate (80 mL×3). The combined organic phase was washed with 15 mL saturated brine, the ethyl acetate phase was dried over sodium sulfate, filtered and concentrated under reduced pressure. The residue was dissolved in the mixture of 30 mL dichloromethane, 10 mL water, ethanedioic acid (1.18 g, 9.35 mL) and then was stirred overnight. The reaction mixture was extracted with ethyl acetate (80 mL×3). The combined organic phase was washed with 15 mL saturated brine, the ethyl acetate phase was dried over anhydrous sodium sulfate, filtered and concentrated under reduced pressure. The residue was purified by silica gel column chromatography to give the title compound 5-(4-fluoro-phenyl)-5-hydroxy-hexahydro-pentalen-2-one 12a (900 mg, yield 60%) as a white powder.

MS m/z (ESI): 235.3 [M+1].

### Preparation of 1-{2-[5-(4-fluoro-phenyl)-5-hydroxy-octahydro-pentalen-2-ylamino]-acetyl}-pyrrolidine-2-carbonitrile 12b

[2-(2-Cyano-pyrrolidin-1-yl)-2-oxo-ethyl]-carbamic acid tert-butyl ester 1f (680 mg, 2.69 mmol) was dissolved in 30 mL then trifluoroacetic acid (6.18 mL, 80.7 mmol) was added in an ice-water bath. Upon completion of the addition, the reaction mixture was stirred at 0° until reaction was completed. Dichloromethane and trifluoroacetic acid were evaporated. The above residue was dissolved in 20 mL methanol and in order added with triethylamine (0.747 mL, 5.37 mmol), 5-(4-fluoro-phenyl)-5-hydroxy-hexahydro-pentalen-2-one 12a (420 mg, 1.79 mmol) and sodium triacetoxy borohydride (1.71 g, 8.06 mmol). The reaction mixture was stirred at room temperature, concentrated under reduced pressure and added with 20 mL saturated sodium carbonate solution, then extracted with dichloromethane (80 mL×3). The combined organic phase was washed with 15 mL saturated brine, the dichloromethane phase was dried over anhydrous sodium sulfate, filtered and concentrated under reduced pressure. The residue was purified by silica gel column chromatography to give the title compound 1-(2-(5-(4-fluoro-phenyl)-5-hydroxy-octahydro-pentalen-2-ylamino)-acetyl)-pyrrolidine-2-carbonitrile 12b (110 mg, yield 16.6%) as a white powder.

MS m/z (ESI): 372.3 [M+1].

¹H NMR (400 MHz, CDCl₃) δ (ppm) 7.44 (m, 2H), 6.97 (m, 2H), 4.75 (m, 1H), 3.64-3.22 (m, 5H), 2.86 (m, 2H), 2.40 (m, 2H), 2.36-2.04 (m, 8H), 1.82 (m, 2H).

### Preparation of 1-{2-[5-(4-fluoro-phenyl)-5-hydroxy-octahydro-pentalen-2-ylamino]-acetyl}-pyrrolidine-2-carbonitrile Hydrochloride 12

1-{2-[5-(4-Fluoro-phenyl)-5-hydroxy-octahydro-pentalen-2-ylamino]-acetyl}-pyrrolidine-2-carbonitrile 12b (110 mg, 0.296 mmol) was dispersed in 10 mL ether, then a solution of 0.5N hydrochloric acid in 2 mL ether was added in an ice-water bath. The resulting solid was centrifuged to give the title compound 1-{2-[5-(4-fluoro-phenyl)-5-hydroxy-octahydro-pentalen-2-ylamino]-acetyl)-pyrrolidine-2-carbonitrile hydrochloride 12 (100 mg, yield 83%) as a white powder.

MS m/z (ESI): 372.4 [M+1].

### Example 13

### Preparation of 1-[2-(5-cyclohexyl-5-hydroxy-octahydro-pentalen-2-ylamino)-acetyl]-pyrrolidine-2-carbonitrile Hydrochloride 13

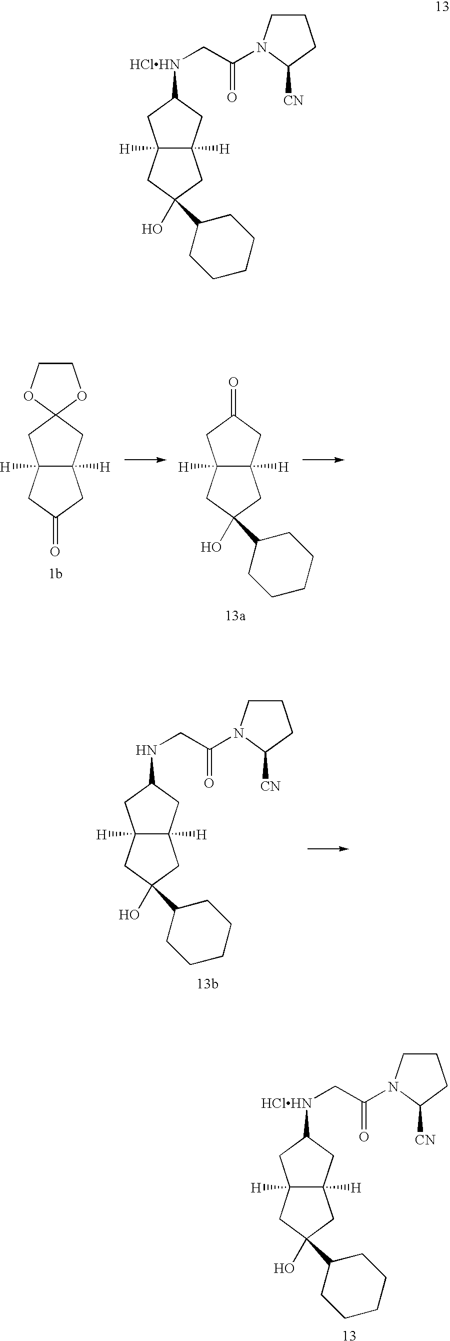

### Preparation of 5-cyclohexyl-5-hydroxy-hexahydro-pentalen-2-one 13a

Magnesium (486 mg, 20 mmol) was added to 10 mL ether and lightly heated. The mixture was stirred and added with catalytic amount iodine and dropwise 1-chloro-hexane (2.48 mL, 21 mmol) while maintaining the reaction system lightly boiled for 1 hour. The reaction mixture was dropwise added with a solution of 7,7-(ethylidene acetal)bicyclo[3.3.0] octane-3-one 1b (0.546 g, 3 mmol) in 10 mL ether and then was stirred for 2 hours, added with 2 mL 2N hydrochloric acid to stop the reaction for another 2 hours stirring and extracted with ethyl acetate (80 mL×3). The combined organic phase was washed with 15 mL saturated brine, the ethyl acetate phase was dried over anhydrous sodium sulfate, filtered and concentrated under reduced pressure. The residue was dissolved in the mixture of 10 mL ethyl acetate, 5 mL water, ethanedioic acid (189 mg, 1.5 mmol) and then stirred for 6 h at 40°. The reaction mixture was extracted with ethyl acetate (80 mL×3). The combined organic phase was washed with 15 mL saturated brine, the ethyl acetate phase was dried over anhydrous sodium sulfate, filtered and concentrated under reduced pressure. The residue was purified by silica gel column chromatography to give the title compound 5-cyclohexyl-5-hydroxy-hexahydro-pentalen-2-one 13a (110 mg, yield 54%) as a light yellow powder.

MS m/z (ESI): 223.1 [M+1].

### Preparation of 1-[2-(5-cyclohexyl-5-hydroxy-hexahydro-pentalen-2-ylamino)-acetyl]-pyrrolidine-2-carbonitrile 13b

[2-(2-Cyano-pyrrolidin-1-yl)-2-oxo-ethyl]-carbamic acid tert-butyl ester 1f (479 mg, 1.89 mmol) was dissolved in 20 mL dichloromethane, then trifluoroacetic acid (4.34 mL, 56.7 mmol) was added in an ice-water bath. Upon completion of the addition, the reaction mixture was stirred at 0° until reaction was completed. Dichloromethane and trifluoroacetic acid were evaporated. The above residue was dissolved in 20 mL methanol, in order added with triethylamine (0.66 mL, 4.74 mmol), 5-cyclohexyl-5-hydroxy-hexahydro-pentalen-2-one 13a (350 mg, 1.58 mmol) and sodium triacetoxy borohydride (0.51 g, 7.11 mmol). The reaction mixture was stirred at room temperature, concentrated under reduced pressure, added with 20 mL saturated sodium carbonate solution, the extracted with dichloromethane (80 mL×3). The combined organic phase was washed with 15 mL saturated brine, the dichloromethane phase was dried over anhydrous sodium sulfate, filtered and concentrated under reduced pressure. The residue was purified by silica gel column chromatography to give the title compound 1-[2-(5-cyclohexyl-5-hydroxy-hexahydro-pentalen-2-ylamino]-acetyl)-pyrrolidine-2-carbonitrile 13b (90 mg, yield 16.0%) as a white powder.

MS m/z (ESI): 360.3 [M+1].

### Preparation of 1-[2-(5-cyclohexyl-5-hydroxy-octahydro-pentalen-2-ylamino)-acetyl]-pyrrolidine-2-carbonitrile hydrochloride 13

1-[2-(5-Cyclohexyl-5-hydroxy-hexahydro-pentalen-2-ylamino)-acetyl]-pyrrolidine-2-carbonitrile 13b (90 mg, 0.28 mmol) was dispersed in 10 mL ether, then a solution of 0.5N hydrochloric acid in 2 mL ether was in an ice-water bath. The reaction mixture was evaporated to give the title compound 1-[2-(5-cyclohexyl-5-hydroxy-octahydro-pentalen-2-ylamino)-acetyl]-pyrrolidine-2-carbonitrile hydrochloride 13 (80 mg, yield 80%) as a white powder.

MS m/z (ESI): 360.2 [M+1].

### Example 14

### Preparation of 3-{2-[5-(4-Fluoro-phenyl)-5-hydroxy-octahydro-pentalen-2-ylamino]-acetyl}-thiazolidine-2-carbonitrile Hydrochloride 14

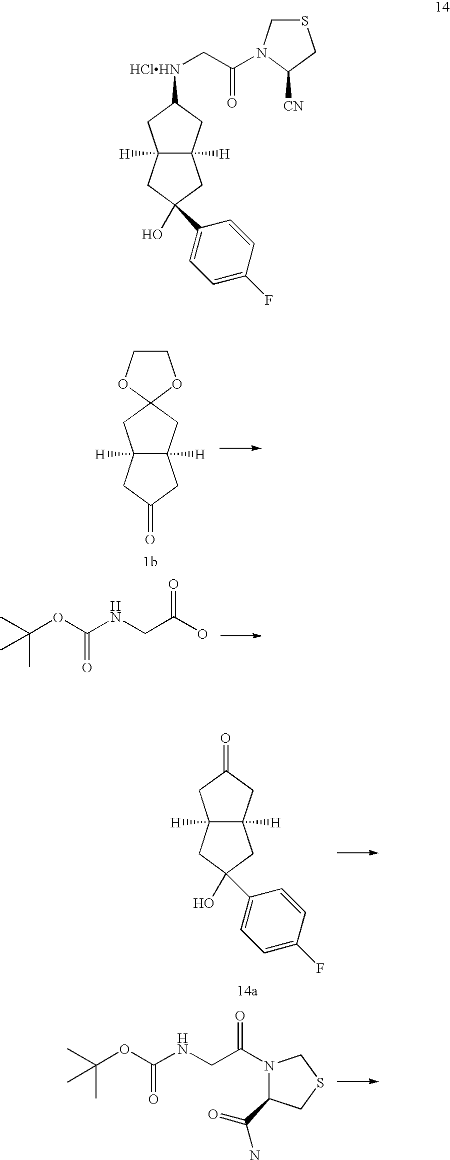

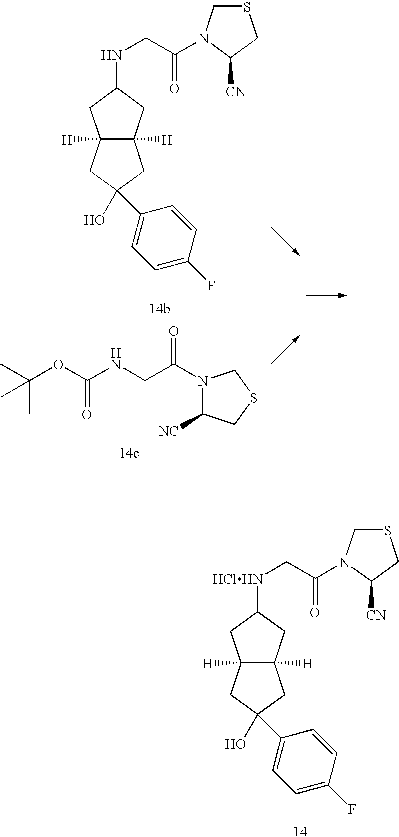

### Preparation of 5-(4-fluoro-phenyl)-5-hydroxy-hexahydro-pentalen-2-one 14a

Magnesium (486 mg, 20 mmol) was added to 20 mL ether and lightly heated. The mixture was stirred and added with catalytic amount iodine and dropwise 1-bromo-4-fluoro-benzene (2.1 mL, 22 mmol) while maintaining the reaction system lightly boiled for 1 hour. The reaction mixture was dropwise added with a solution of 7,7-(ethylidene acetal)bicyclo[3.3.0]octane-3-one 1b (1.19 g, 6.54 mmol) in 10 mL ether and then was stirred for 2 hours, added with 2 mL 2N hydrochloric acid to stop the reaction for another 2 hours stirring and extracted with ethyl acetate (80 mL×3). The combined organic phase was washed with 15 mL saturated brine, the ethyl acetate was dried over anhydrous sodium sulfate, filtered and concentrated under reduced pressure. The residue was purified by silica gel column chromatography to give the title compound 5-(4-fluoro-phenyl)-5-hydroxy-hexahydro-pentalen-2-one 14a (900 mg, yield 60%) as a white powder.

MS m/z (ESI): 235.3 [M+1].

### Preparation of tert-butyl-[2-(4-carbamoyl-thiazolidin-3-yl)-2-oxo-ethyl]-carbamate 14b

N-tert-buthylcarbonyl-glycine (0.75 g, 4.31 mmol) and R-thiazolidine-4-carboxamide (1.05 g, 4.31 mmol) were dissolved in 75 mL acetonitrile at 0°, then 1-hydroxybenzotriazole (1.74 g, 12.93 mmol), N-ethyl-N′-(dimethylaminopropane)-carbodiimide (1.65 g, 8.62 mmol) and triethylamine (1.8 mL, 12.93 mmol) were added under stirring. Upon completion of the addition, the reaction mixture was naturally raised to room temperature and stirred for 3 hours. After TLC showed the starting material disappeared, the redundant acenitrile was removed and the reaction mixture was extracted with ethyl acetate (100 mL×3). The combined organic phase was washed with 25 mL saturated brine, dried over anhydrous sodium sulfate, filtered and concentrated under reduced pressure. The residue was purified by silica gel column chromatography to give the title compound tert-butyl-[2-(4-carbamoyl-thiazolidin-3-yl)-2-oxo-ethyl]-carbamate 14b (1.15 g, yield 92.7%) as a white powder.

MS m/z (ESI): 290.1 [M+1].

### Preparation of tert-butyl-[2-(4-cynao-thiazolidin-3-yl)-2-oxo-ethyl]-carbamate 14c

In a dry three-neck flask under a nitrogen atmosphere, 20 mL pyridine, tert-butyl-[2-(4-carbamoyl-thiazolidin-3-yl)-2-oxo-ethyl]-carbamate 14b (1.15 g, 3.98 mmol), imidazole (0.57 g, 8.36 mmol) was added in order while maintaining the reaction system at −35°, phosphorus oxychloride (1.52 mL, 16.32 mmol) was added dropwise under stirring for 1 hour at −35°. The reaction mixture was naturally raised to room temperature, aboundent pyridine was evaporated and the reaction mixture was extracted with ethyl acetate (100 mL×3). The combined organic phase was washed with 25 mL saturated brine, dried over anhydrous sodium sulfate, filtered and concentrated under reduced pressure. The residue was purified by silica gel column chromatography to give the title compound tert-butyl-[2-(4-cynao-thiazolidin-3-yl)-2-oxo-ethyl]-carbamate 14c (0.811 g, yield 75%) as a white powder.

MS m/z (ESI): 272.3 [M+1].

### Preparation of 3-{2-[5-(4-Fluoro-phenyl)-5-hydroxy-octahydro-pentalen-2-ylamino]-acetyl}-thiazolidine-2-carbonitrile 14d

Tert-butyl-[2-(4-cynao-thiazolidin-3-yl)-2-oxo-ethyl]-carbamate 14c (698 mg, 2.58 mmol) was dissolved in 20 mL dichloromethane, then trifluoroacetic acid (5.93 mL, 77.4 mmol) was added in an ice-water bath. Upon completion of the addition, the reaction mixture was stirred at 0° until reaction was completed. Dichloromethane and trifluoroacetic acid were evaporated. The above residue was dissolved in 15 mL methanol and in order added with triethylamine (1.04 mL, 7.5 mmol), 5-(4-fluoro-phenyl)-5-hydroxy-hexahydro-pentalen-2-one 14a (600 mg, 2.5 mmol) and sodium triacetoxy borohydride (2.38 g, 11.25 mmol). The reaction mixture was stirred at room temperature, concentrated under reduced pressure and added with 20 mL saturated sodium carbonate solution, then extracted with dichloromethane (80 mL×3). The combined organic phase was washed with 15 mL saturated brine, the dichloromethane was dried over anhydrous sodium sulfate, filtered and concentrated under reduced pressure. The residue was purified by silica gel column chromatography to give the title compound 3-{2-[5-(4-Fluoro-phenyl)-5-hydroxy-octahydro-pentalen-2-ylamino]-acetyl}-thiazolidine-2-carbonitrile 14d (80 mg, yield 19.6%) as a light yellow powder.

MS m/z (ESI): 390.3 [M+1].

¹H NMR (400 MHz, CD₃OD) δ (ppm) 7.35 (m, 2H), 6.89 (m, 2H), 4.62 (m, 1H), 4.39 (m, 2H), 3.83 (m, 1H), 3.39 (m, 1H), 3.18 (m, 3H), 3.10 (m, 1H), 2.62 (m, 2H), 2.06 (m, 2H), 2.00-1.72 (m, 5H).

### Preparation of 3-{2-[5-(4-Fluoro-phenyl)-5-hydroxy-octahydro-pentalen-2-ylamino]-acetyl}-thiazolidine-2-carbonitrile Hydrochloride 14

3-{2-[5-(4-Fluoro-phenyl)-5-hydroxy-octahydro-pentalen-2-ylamino]-acetyl}-thiazolidine-2-carbonitrile 14d (80 mg, 0.206 mmol) was dispersed in 10 mL ether, then a solution of 0.5N hydrochloric acid in 2 mL ether was added in an ice-water bath. The reaction mixture was evaporated to give the title compound 3-{2-[5-(4-Fluoro-phenyl)-5-hydroxy-octahydro-pentalen-2-ylamino]-acetyl}-thiazolidine-2-carbonitrile hydrochloride 14 (80 mg, yield 91%) as a white powder.

MS m/z (ESI): 390.2 [M+1].

### Example 15

### Preparation of 1-[2-(5-oxo-octahydro-pentalen-2-ylamino)-acetyl]-pyrrolidine-2-carbonitrile Hydrochloride 15

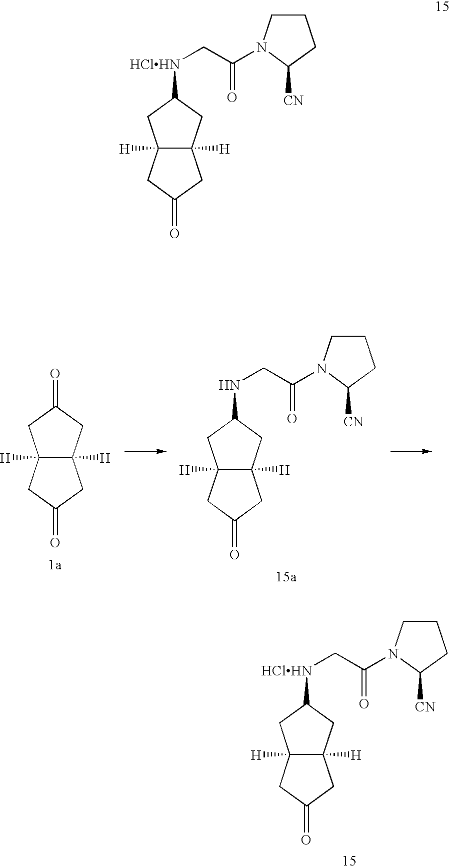

### Preparation of 1-[2-(5-oxo-octahydro-pentalen-2-ylamino)-acetyl]-pyrrolidine-2-carbonitrile 15a

[2-(2-Cyano-pyrrolidin-1-yl)-2-oxo-ethyl]-carbamic acid tert-butyl ester (1.83 g, 7.246 mmol) was dissolved in 40 mL dichloromethane, then trifluoroacetic acid (16.65 mL, 217.4 mmol) was added in an ice-water bath. Upon completion of the addition, the reaction mixture was stirred at 0° until reaction was completed. Dichloromethane and trifluoroacetic acid were evaporated. The above residue was dissolved in 40 mL methanol and in order added with triethylamine (3.024 mL, 21.74 mmol), tetrahydro-pentalene-2,5-dione 1a (1 g, 7.246 mmol) and sodium triacetoxy borohydride (6.142 g, 28.98 mmol). The reaction mixture was stirred overnight at room temperature, concentrated under reduced pressured and added with 10 mL saturated sodium carbonate solution, then extracted with dichloromethane (80 mL×3). The combined organic phase was washed with 15 mL saturated brine, the dichloromethane phase was dried over anhydrous sodium sulfate, filtered and concentrated under reduced pressure. The residue was purified by silica gel column chromatography to give the title compound 1-[2-(5-oxo-octahydro-pentalen-2-ylamino)-acetyl]-pyrrolidine-2-carbonitrile 15a (100 mg, yield 5%) as a white powder.

MS m/z (ESI): 276.6 [M+1].

¹H NMR (400 MHz, CDCl₃) δ (ppm) 4.69 (m, 1H), 3.52 (m, 1H), 3.36 (m, 1H), 3.16 (m, 1H), 2.66 (m, 2H), 2.44 (m, 3H), 2.24-1.98 (m, 8H), 1.25 (m, 3H).

### Preparation of 1-[2-(5-oxo-octahydro-pentalen-2-ylamino)-acetyl]-pyrrolidine-2-carbonitrile Hydrochloride 15

1-[2-(5-Oxo-octahydro-pentalen-2-ylamino)-acetyl]-pyrrolidine-2-carbonitrile 15a (100 mg, 0.364 mmol) was dispersed in 10 mL ether, then a solution of 0.5N hydrochloric acid in 2 mL ether was added in an ice-water bath. The resulting solid was centrifuged to give the title compound 1-[2-(5-oxo-octahydro-pentalen-2-ylamino)-acetyl]-pyrrolidine-2-carbonitrile hydrochloride 15 (80 mg, yield 70.1%) as a white powder.

MS m/z (ESI): 276.2 [M+1].

### Example 16

### Preparation of 1-[2-(5-hydroxyl-octahydro-pentalen-2-ylamino) acetyl]pyrrolidine-2-carbonitrile Hydrochloride 16

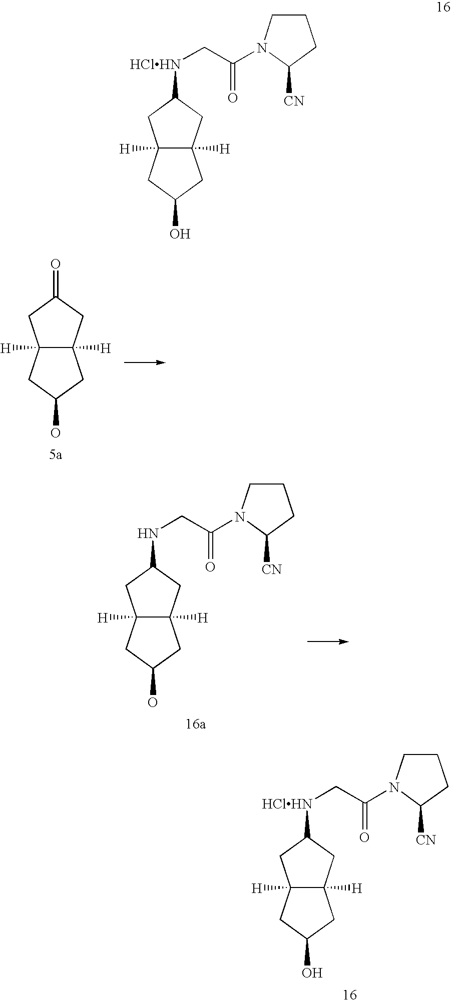

### Preparation of 1-[2-(5-hydroxyl-octahydro-pentalen-2-ylamino)acetyl]pyrrolidine-2-carbonitrile 16a

[2-(2-Cyano-pyrrolidin-1-yl)-2-oxo-ethyl]-carbamic acid tert-butyl ester 1f (903 mg, 3.571 mmol) was dissolved in 40 mL dichloromethane, then trifluoroacetic acid (8.2 mL, 107.14 mmol) was added in an ice-water bath. Upon completion of the addition, the reaction mixture was stirred at 0° until reaction was completed. Dichloromethane and trifluoroacetic acid were evaporated. The above residue was dissolved in 50 mL methanol and in order added with triethylamine (1.49 mL, 10.714 mmol), 5-hydroxy-hexahydro-pentalen-2-one 5a (0.5 g, 3.571 mmol) and sodium triacetoxy borohydride (3.02 g, 14.285 mmol). The reaction mixture was stirred overnight at room temperature, concentrated under reduced pressure and added with 20 mL saturated sodium carbonate solution, then extracted with ethy acetate (80 mL×3). The combined organic phase was washed with 15 mL saturated brine, the ethyl acetate was dried over anhydrous sodium sulfate, filtered and concentrated under reduced pressure. The residue was purified by silica gel column chromatography to give the title compound 1-[2-(5-hydroxyl-octahydro-pentalen-2-ylamino)acetyl]pyrrolidine-2-carbonitrile 16a (200 mg, yield 20.4%) as a white powder.

MS m/z (ESI): 278.5 [M+1].

¹H NMR (400 MHz, CDCl₃) δ (ppm) 4.49 (m, 1H), 3.98 (m, 1H), 3.36 (m, 2H), 3.20 (m, 1H), 3.07 (s, 1H), 2.99 (m, 1H), 2.77 (m, 2H), 1.98 (m, 5H), 1.79 (m, 2H), 1.31 (m, 3H), 1.05 (m, 2H).

### Preparation of 1-[2-(5-hydroxyl-octahydro-pentalen-2-ylamino) acetyl]pyrrolidine-2-carbonitrile Hydrochloride 16

1-[2-(5-Hydroxyl-octahydro-pentalen-2-ylamino)acetyl]pyrrolidine-2-carbonitrile 16a (192 mg, 0.686 mmol) was dispersed in 10 mL ether, then a solution of 0.5N hydrochloric acid in 3 mL ether was added in an ice-water bath. The resulting solid was centrifuged to give the title compound 1-(2-(5-hydroxyl-octahydro-pentalen-2-ylamino)-acetyl)pyrrolidine-2-carbonitrile hydrochloride 16 (80 mg, yield 40%) as a white powder.

MS m/z (ESI): 278.2 [M+1].

### Example 17

### Preparation of 1-[2-(5-methoxy-octahydro-pentalen-2-ylamino)-acetyl]-pyrrolidine-2-carbonitrile Hydrochloride

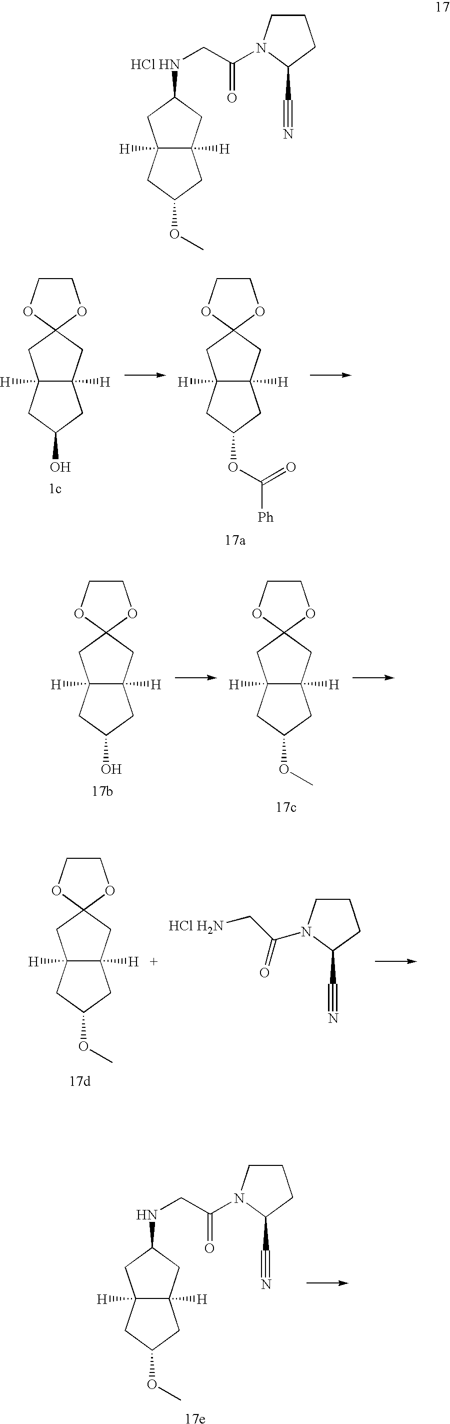

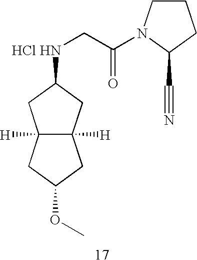

### Preparation of 7,7-(ethylidene acetal)bicyclo[3.3.0] octane-3-benzoate 17a

A solution of benzoic acid (2.68 g, 22 mmol) and diethyl azodicarboxylate (3.828 g, 22 mmol) in 4 mL ether was added with a solution of 3-hydroxy-7,7-(ethylidene acetal)bicyclo[3.3.0] octane 1c (3.68 g, 20 mmol) and triphenylphosphine (5.786 g, 22 mmol) in 50 mL ether. Upon completion of the addition, the reaction mixture was stirred for 14 hours at room temperature, concentrated. The residue was purified by silica gel column chromatography to give the title compound 7,7-(ethylidene acetal)bicyclo[3.3.0] octane-3-benzoate 17a (2.9 g, yield 50%) as a colorless oil.

### Preparation of 3-hydroxy-7,7-(ethylidene acetal)bicyclo[3.3.0] octane 17b

7,7-(Ethylidene acetal)bicyclo[3.3.0] octane-3-benzoate 17a (2.9 g, 10.0 mmol) and potassium hydroxide (3.2 g, 22 mmol) were dissolved in a mixture of 73 mL methanol and 37 mL water, the reaction mixture was stirred for 2 hours at 40° C. The reaction mixture was extracted with acetic ether (50 mL×4). The combined organic phase was dried over anhydrous sodium sulfate, filtered and concentrated under reduced pressure to give the title compound 3-hydroxy-7,7-(ethylidene acetal)bicyclo[3.3.0] octane 17b (1.7 g, yield 93%) as a colorless oil.

MS m/z (ESI): 185.5 [M+1].

### Preparation of 3-methoxy-7,7-(ethylidene acetal)bicyclo[3.3.0] octane 17c

3-hydroxy-7,7-(ethylidene acetal)bicyclo[3.3.0] octane 17b (0.86 g, 4.67 mmol) was dissolved in 50 mL tetrahydrofuran, then sodium hydride (0.45 g, 9.34 mmol) was added. Upon completion of the addition, the reaction mixture was stirred for 1 hour at room temperature, added with methyl iodide (1.162 mL, 18.69 mmol) and heated to reflux for 2 hours, cooled and added with water (10 mL) to stop the reaction, extracted with ethyl acetate (50 mL×4). The combined organic phase was dried over anhydrous sodium sulfate, filtered and concentrated under reduced pressure to give the title compound 3-methoxy-7,7-(ethylidene acetal)bicyclo[3.3.0] octane 17c to the next step.

### Preparation of 5-methoxy-hexahydropentalen-2-one 17d

3-Methoxy-7,7-(ethylidene acetal)bicyclo[3.3.0] octane 17c (920 mg, 4.67 mmol) was dissolved in the mixture of 50 mL ethyl acetate and 25 mL water, then ethanedioic acid (1.134 g, 9 mmol) was added under stirring. Upon completion of the addition, the reaction mixture was stirred overnight at room temperature, added with 50 mL water, extracted with ethyl acetate (50 mL×3). The combined organic phase was dried over anhydrous sodium sulfate, filtered and concentrated under reduced pressure to give the title compound 5-methoxy-hexahydropentalen-2-one 17d (0.65 g) as a colorless oil.

MS m/z (ESI): 157.3 [M+1].

### Preparation of 1-[2-(5-methoxy-octahydro-pentalen-2-ylamino)-acetyl]-pyrrolidine-2-carbonitrile 17e

5-Methoxy-hexahydropentalen-2-one 17d (308 mg, 2 mmol) was dissolved in 50 mL tetrahydrofuran, then 1-(2-Amino-acetyl)-pyrrolidine-2-carbonitrile hydrochloride (630 mg, 3.32 mmol) was added. The reaction mixture was stirred for 0.5 hour, and then added with sodium sulfate (5 g), sodium triacetoxy borohydride (1.4 g, 6.6 mmol). Upon completion of the addition, the reaction mixture was stirred for 3 hours at room temperature, added with 20 mL saturated sodium carbonate solution, extracted with ethyl acetate (50 mL×5), extracted with dichloromethane (50 mL×10). The combined organic phase was washed with 50 mL saturated brine, dried over anhydrous sodium sulfate, filtered and concentrated under reduced pressure. The residue was purified by silica gel column chromatography to give the title compound 1-[2-(5-methoxy-octahydro-pentalen-2-ylamino)-acetyl]-pyrrolidine-2-carbonitrile 17e (250 mg, yield 43%) as a colorless oil.

MS m/z (ESI): 320.3 [M+1].

### Preparation of 1-[2-(5-methoxy-octahydro-pentalen-2-ylamino)-acetyl]-pyrrolidine-2-carbonitrile Hydrochloride 17

1-[2-(5-Methoxy-octahydro-pentalen-2-ylamino)-acetyl]-pyrrolidine-2-carbonitrile 17e (250 mg, 0.313 mmol) was dispersed in 10 mL ether, then a 2 mL solution of 0.5N hydrochloric acid in ether was added in an ice-water bath. The resulting solid was centrifuged to give the title compound 1-[2-(5-methoxy-octahydro-pentalen-2-ylamino)-acetyl]-pyrrolidine-2-carbonitrile hydrochloride 17 (250 mg, yield 76.3%) as a white powder.

MS m/z (ESI): 292.6 [M+1].

### Example 18

### Preparation of 1-[2-(5-methoxy-octahydro-pentalen-2-ylamino)-acetyl)]pyrrolidine-2-carbonitrile Hydrochloride 18

Error! Objects cannot be created from editing field codes.

Error! Objects cannot be created from editing field codes.

### Preparation of 5-methoxy-octahydro-pentalen-2-ol 18a

5-Methoxy-hexahydropentalen-2-one 17d (2.5 g, 7.12 mmol) was dissolved in 50 mL methanol, then sodium borohydride (0.537 g, 14.2 mmol) was added in an ice-water bath. Upon completion of the addition, the reaction mixture was stirred for 30 min, and then added with 10 mL water and small volume acetone (2 mL) to stop the reaction, concentrated under reduced pressure, extracted with acetic ether (50 mL×3). The combined organic phase was dried over anhydrous sodium sulfate, filtered and concentrated under reduced pressure to give the title compound 5-methoxy-octahydro-pentalen-2-ol 18a (1.46 g) as a colorless oil to be directly used in the further reaction.

### Preparation of methanesulfonic acid 5-methoxy-octahydro-pentalen-2-yl ester 18b

5-Methoxy-octahydro-pentalen-2-ol 18a (1.47 g, 7.0 mmol) was dissolved in 50 mL dichlormethane, and triethylamine (1.95 ml, 14.0 mmol) was added, then methanesulfonyl chloride (0.7 ml, 9.1 mmol) was added in an ice-water bath. Upon completion of the addition, the reaction mixture was stirred overnight, added with 50 mL water, extracted with dichlormethane (50 mL×4). The combined organic phase was dried over anhydrous sodium sulfate, filtered and concentrated under reduced pressure, the residue was purified by silica gel column chromatography to give the title compound methanesulfonic acid 5-methoxy-octahydro-pentalen-2-yl ester 18b (1.13 g, yield 69.0%) as a colorless oil.

### Preparation of 5-methoxy-octahydro-pentalen-2-yl azide group 18c

Methanesulfonic acid 5-methoxy-octahydro-pentalen-2-yl ester 18b (1.13 g, 4.82 mmol), sodium azide (0.313 g, 4.82 mmol) were dissolved in 50 mL N,N-dimethylformamide under an argon atmosphere, the reaction mixture was heated to 65° C. overnight. The resulting mixture was cooled, concentrated, added with 50 mL water, then extracted with ethyl acetate (50 mL×4) and The combined organic phase was dried over anhydrous sodium sulfate, filtered and concentrated under reduced pressure. The residue was purified by silica gel column chromatography to give 5-methoxy-octahydro-pentalen-2-yl azide group 18c (500 mg, yield 57%) as a colorless oil.

### Preparation of 5-methoxy-octahydro-pentalen-2-ylamine 18d

5-Methoxy-octahydro-pentalen-2-yl azide group 18c (0.15 g, 0.83 mmol) was dissolved in 50 mL methanol, Pd—C (0.05 g) and chloroform (0.1 ml) was added. Upon completion of the addition, the reaction mixture was hydrogenated for 3 hours under 0.3 Mpa at room temperature, filtered and washed with methanol (10 mL×5). The filtrate was evaporated to give the title compound 5-methoxy-octahydro-pentalen-2-ylamine 18d (0.16 g) to be used directly in the next step.

MS m/z (ESI): 156.5 [M+1].

### Preparation of 1-[2-(5-methoxy-octahydro-pentalen-2-ylamino)-acetyl]-pyrrolidine-2-carbonitrile 18e

5-Methoxy-octahydro-pentalen-2-ylamine 18d (0.16 g, 0.83 mmol), 1-(2-chloro-acetyl)-pyrrolidine-2-carbonitrile (143 mg, 0.83 mmol) [J. Med. Chem. 2002 45(12), 2362-2365], potassium carbonate (0.230 g, 1.66 mmol) and catalytic amount potassium iodide were dissolved in 100 mL acetonitrile. Upon completion of the addition, the reaction mixture was stirred overnight at room temperature, added with 20 mL water, adjusted to pH >10 with 1N sodium hydroxide solution and extracted with ethyl acetate (50 ml×8) while maintaining pH >10 in water phase. The combined organic phase was washed with saturated brine (50 mL), dried over anhydrous sodium sulfate, filtered and concentrated under reduced pressure. The residue was purified by silica gel column chromatography to give the title compound 1-[2-(5-methoxy-octahydro-pentalen-2-ylamino)-acetyl]-pyrrolidine-2-carbonitrile 18e (0.142 g) as a colorless oil to be directly used in the further step.

MS m/z (ESI): 292.6 [M+1].

### Preparation of 1-[2-(5-methoxy-octahydro-pentalen-2-ylamino)-acetyl]-pyrrolidine-2-carbonitrile Hydrochloride 18

1-[2-(5-Methoxy-octahydro-pentalen-2-ylamino)-acetyl]-pyrrolidine-2-carbonitrile 18e (142 mg, 0.487 mmol) was dispersed in 10 mL ether, then a 2 mL solution of 0.5N hydrochloric acid in ether was added in an ice-water bath. The resulting solid was centrifuged to give the title compound 1-[2-(5-methoxy-octahydro-pentalen-2-ylamino)-acetyl]-pyrrolidine-2-carbonitrile hydrochloride 18 (142 mg, yield 89%) as a white powder.

### Example 19

### Preparation of 1-[2-(5-methoxy-octahydro-pentalen-2-ylamino)-acetyl]-pyrrolidine-2-carbonitrile Hydrochloride 19

Error! Objects cannot be created from editing field codes.

Error! Objects cannot be created from editing field codes.

### Preparation of 5-methoxy-octahydro-pentalen-2-ol 19a

5-Methoxy-hexahydropentalen-2-one 1e (2.5 g, 7.12 mmol) was dissolved in 50 mL methanol, then sodium borohydride (0.537 g, 14.2 mmol) was added in an ice-water bath. Upon completion of the addition, the reaction mixture was stirred for 30 min, then added with 10 mL water and small volume acetone (2 mL) to stop the reaction, concentrated under reduced pressure, extracted with acetic ether (50 mL×3). The combined organic phase was dried over anhydrous sodium sulfate, filtered and concentrated under reduced pressure to give the title compound 5-methoxy-octahydro-pentalen-2-ol 19a (1.46 g) as a colorless oil to be directly used in the further reaction.

### Preparation of methanesulfonic acid 5-methoxy-octahydro-pentalen-2-yl Ester 19b

5-Methoxy-octahydro-pentalen-2-ol 19a (1.46 g, 7.12 mmol) was dissolved in 50 mL dichlormethane, and triethylamine (1.98 ml, 14.2 mmol) was added, then methanesulfonyl chloride (0.66 ml, 8.54 mmol) was added in an ice-water bath. Upon completion of the addition, the reaction mixture was stirred overnight, added with 50 mL water, extracted with dichlormethane (50 mL×4). The combined organic phase was dried over anhydrous sodium sulfate, filtered and concentrated under reduced pressure, the residue was purified by silica gel column chromatography to give the title compound methanesulfonic acid 5-methoxy-octahydro-pentalen-2-yl ester 19b (1.47 g, yield 88.1%) as a colorless oil.

### Preparation of 5-methoxy-octahydro-pentalen-2-yl Azide Group 19c

Methanesulfonic acid 5-methoxy-octahydro-pentalen-2-yl ester 19b (1.47 g, 6.27 mmol), sodium azide (0.408 g, 6.27 mmol) were dissolved in 50 mL N,N-dimethylformamide under an argon atmosphere, the reaction mixture was heated to 65° C. overnight. The resulting mixture was cooled, concentrated under reduced pressure, added with 50 mL water and extracted with ethyl acetate (50 mL×4). The combined organic phase was dried over anhydrous sodium sulfate, filtered and concentrated under reduced pressure. The residue was purified by silica gel column chromatography to give the title compound 5-methoxy-octahydro-pentalen-2-yl azide group 19c (540 mg, yield 48%) as a colorless oil.

### Preparation of 5-methoxy-octahydro-pentalen-2-ylamine 19d

5-Methoxy-octahydro-pentalen-2-yl azide group 19c (0.52 g, 2.87 mmol) was dispersed in 20 mL methanol, then Pd—C (0.25 g) was added. Upon completion of the addition, the reaction mixture was hydrogenated for 3 hours under 0.3 Mpa at room temperature, filtered and Pd—C was washed with methanol (10 mL×5). The filtrate was concentrated to give the title compound 5-methoxy-octahydro-pentalen-2-ylamine 19d (0.42 g, yield 92.2%).

MS m/z (ESI): 156.5 [M+1].

### Preparation of 1-[2-(5-methoxy-octahydro-pentalen-2-ylamino)-acetyl]-pyrrolidine-2-carbonitrile 19e

5-Methoxy-octahydro-pentalen-2-ylamine 19d (0.42 g, 1.3 mmol), 1-(2-chloro-acetyl)-pyrrolidine-2-carbonitrile (334 mg, 1.9 mmol) [J. Med. Chem. 2002 45(12), 2362-2365], potassium carbonate (0.719 g, 5.2 mmol) and potassium iodide (100 mg, 0.6 mmol) was dissolved in 20 mL dichlormethane. Upon completion of the addition, the reaction mixture was stirred overnight at room temperature, added with 20 mL water, adjusted to pH >10 with 1N sodium hydroxide solution and extracted with ethyl acetate (50 ml×8) while maintaining pH >10 in water phase. The combined organic phase was washed with saturated brine (50 mL), dried over anhydrous sodium sulfate, filtered and concentrated under reduced pressure. The residue was purified by silica gel column chromatography to give the title compound 1-[2-(5-methoxy-octahydro-pentalen-2-ylamino)-acetyl]-pyrrolidine-2-carbonitrile 19e (0.2 g, yield 35%) as a colorless oil.

MS m/z (ESI): 292.6 [M+1].

### Preparation of 1-[2-(5-methoxy-octahydro-pentalen-2-ylamino)-acetyl]-pyrrolidine-2-carbonitrile Hydrochloride 19

1-[2-(5-Methoxy-octahydro-pentalen-2-ylamino)-acetyl]-pyrrolidine-2-carbonitrile 19e (200 mg, 0.687 mmol) was dispersed in 10 mL ether, then a 2 mL solution of 0.5N hydrochloric acid in ether was added in an ice-water bath. The resulting solid was centrifuged to give the title compound 1-[2-(5-methoxy-octahydro-pentalen-2-ylamino)-acetyl]-pyrrolidine-2-carbonitrile hydrochloride 19 (180 mg, yield 80%) as a white powder.

### Biological Assays

### Active Inhibition DPP IV Assay

Aim directly to target DPP IV, Detection method is chemical Luminescent assay, using DPP IV-Glo™ Protease Assay Kit (cat. G8350) of Promega company and Dipeptidylpeptidase IV, Human Placenta (cat. 317630) of Calbiochem company. According to the manual of the kit, this invention inspected the amount of the each reagent in the experimental, corresponding amount of DPPIV enzyme and chemiluminescence detection methods.

The invention inspected the IC₅₀ of the test samples for two parallel treatment, the inhibition rate of compounds for DPP IV is measured and showed in table 1.

TABLE 1
Compounds for DPP IV inhibition assay results

| Example | Structure | IC₅₀(μM) |
|---|---|---|
| 2 | 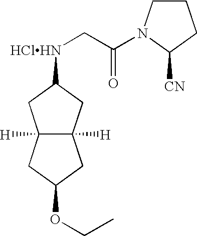 | 0.1269 |
| 3 | 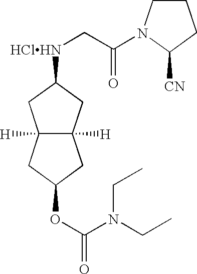 | 0.0405 |
| 4 | 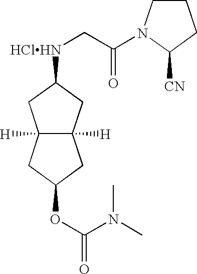 | 0.0613 |
| 5 | 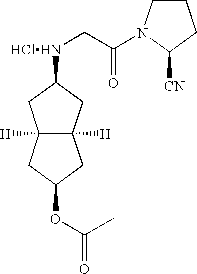 | 0.0322 |
| 6 | 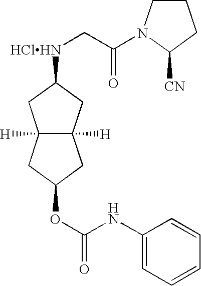 | 0.1211 |
| 7 | 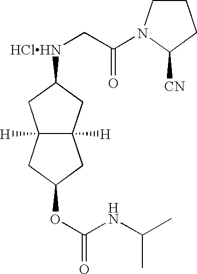 | 0.2036 |
| 8 | 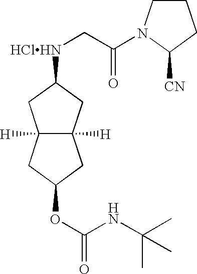 | 0.3257 |
| 10 | 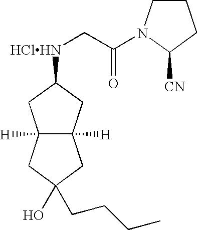 | 0.0183 |
| 14 | 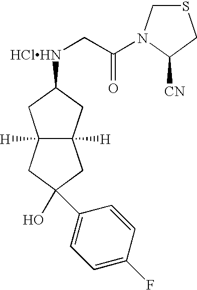 | >2 |

### Preliminary Evaluation on Hypoglycemic Activity of DPPIV Inhibitors

Effects on glucose tolerance in normal ICR mice of examples 15 and 16 were determined. Hypoglycemic activities in vivo were preliminary evaluated.

### Test Drugs:

Preparation procedure: drugs were accurately weighed and dissolved in double-distilled water, misce bene to obtain 0.5 mg/ml suspension, then diluted into colorless and clear solutions which concentration were 0.15, 0.05 and 0.015 mg/ml.

Administrated Dose: the oral doses were 0.3, 1, 3, 10 mg/kg and the volume was 20 ml/kg.

### Positive Control:

Name: LAF-237

Preparation procedure: drugs were accurately weighed and dissolved in double-distilled water, misce bene to obtain 0.5 mg/ml suspension, then diluted into colorless and clear solutions which concentration were 0.15, 0.05 and 0.015 mg/ml. The oral doses were 0.3, 1, 3, 10 mg/kg and the volume was 20 ml/kg.

### Serum Glucose Determination:

Glucose kits was used in the determination of serum glucose levels. Take 250 μl enzyme fluid, then add 5 μl serum. Establish blank tube (by added 5 μl double-distilled) and the standard tube (by added 5 μl standard glucose solution) simultaneously, misce bene. 37° C. water bath for 20 min. To adjust to zero value with blank tube, OD(505 nm) levels were determined.

Serum glucose level BG(mmol/l)=OD_{sample tube}/OD_{standard tube}×5.55.

### Conclusion:

It was found that both examples 15 and 16 have good inhibitor activities of DPPIV in vivo, wherein example 16 is slightly better than LAF-237.

1. Compounds of a Formula (I) or pharmaceutically acceptable salts thereof: 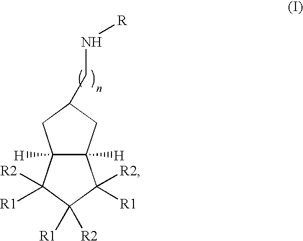
wherein:
R is selected from the group consisting of alkyl, cycloalkyl, haloalkyl, aryl, heteroaryl, aminocarbonyl alkyl, amide alkyl, heterocyclo aminocarbonyl alkyl and aminoalkyl, wherein the heterocycle is selected from the group consisting of 5- or 6-membered hetero ring further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester and halogen;
R₁ and R₂ are each independently selected from the group consisting of hydrogen, hydroxyl, alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;
wherein R₁ and R₂ are attached together with the atom to form a 3 to 8 membered ring, wherein the 3 to 8 membered hetero ring further contains one or more heteroatoms selected from the group consisting of N, O and S, and the 3 to 8 membered hetero rings so formed are further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;
R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;
R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered rings so formed are further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;
R₆ is selected from the group consisting of hydrogen or alkyl;
n is an integer from 0 to 4;
r is an integer from 1 to 6; and
m is an integer from 0 to 6.

2. The compounds or pharmaceutically acceptable salts thereof of claim 1, wherein the compounds include the Formula (IA): 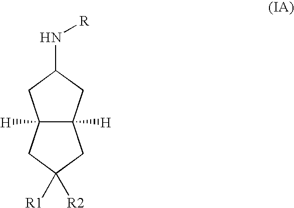
wherein:
R is selected from the group consisting of alkyl, cycloalkyl, haloalkyl, aryl, heteroaryl, aminocarbonyl alkyl, amide alkyl, heterocyclo aminocarbonyl alkyl and aminoalkyl, wherein the heterocycle is selected from the group consisting of 5- or 6-membered hetero ring further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester and halogen;
R₁ and R₂ are each independently selected from the group consisting of hydrogen, hydroxyl, alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;
wherein R₁ and R₂ are attached together with the atom to form a 3 to 8 membered ring, wherein the 3 to 8 membered hetero ring further contains one or more heteroatoms selected from the group consisting of N, O and S, and the 3 to 8 membered hetero rings so formed are each substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;
R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;
R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered hetero ring so formed are further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;
R₆ is selected from the group consisting of hydrogen or alkyl;
r is an integer from 1 to 6; and
m is an integer from 0 to 6.

3. The compounds or pharmaceutically acceptable salts thereof of claim 1, wherein:
R is heterocyclo aminocarbonyl alkyl, wherein the heterocycle is selected from the group consisting of 5- or 6-membered hetero ring further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester and halogen;
R₁ is hydrogen or hydroxyl;
R₂ is selected from the group consisting of hydrogen, hydroxyl, alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkylamino, amide group, alkoxyl, aryloxyl, heterocylco alkyl, carboxylic acid and carboxylic ester;
R₄ and R₅ are each independently selected from the group consisting of hydrogen, alkyl, cycloalkyl, aryl, heteroaryl and heterocyco alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;
R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered hetero rings so formed are substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;
R₆ is selected from the group consisting of hydrogen or alkyl;
r is an integer from 1 to 6; and
m is an integer from 0-6.

4. The compounds or pharmaceutically acceptable salts thereof of claim 1, wherein R is the formula: 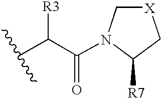
wherein R₃ is selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups selected from the group consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, carboxylic acid and carboxylic ester;
R₇ is selected from the group consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heteroalkyl, carboxylic acid, carboxylic ester and halogen; and
X is C, S or O.

5. The compounds or pharmaceutically acceptable salts thereof of claim 1, wherein n is 0.

6. The compounds or pharmaceutically acceptable salts thereof of claim 1, wherein the compounds of formula (I) are in the pharmaceutically acceptable free-form and the forms of acid addition salts, wherein the salts comprise the salts formed with the acids selected from the group consisting of hydrochloric acid, methanesulfonic acid, sulfuric acid, phosphoric acid, citric acid, acetic acid and trifluoroacetic acid.

7. The compounds or pharmaceutically acceptable salts thereof of claim 6, wherein the acids are hydrochloric acid or trifluoroacetic acid.

8. A preparation process for the compounds of formula (I) of claim 1, wherein the preparation process comprises the steps of: 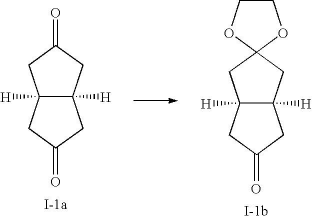
reacting starting material tetrahydro-pentalene-2,5-dione (I-1a) with ethylene glycol and a catalyst of p-toluenesulfonic acid through refluxing in the solvent of benzene to give 7,7-(ethylidene acetal)bicyclo[3.3.0] octane-3-one protected (I-1b); 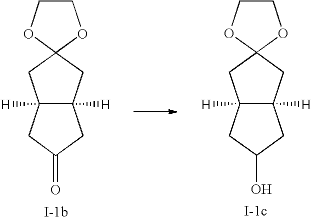
reducing the obtained 7,7-(ethylidene acetal)bicyclo[3.3.0] octane-3-one (I-1b) by NaBH₄ at room temperature to afford 3-hydroxy-7,7-(ethylidene acetal)bicyclo[3.3.0] octane (I-1c); 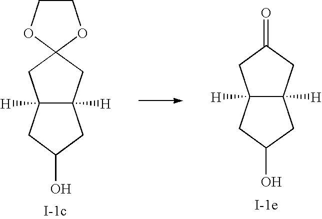
reacting the obtained 3-hydroxy-7,7-(ethylidene acetal)bicyclo[3.3.0] octane (I-1c) with oxalic acid in the mixed solvent of ethyl acetate and water to give 5-hydroxy-hexahydro-pentalen-2-one (I-1e); 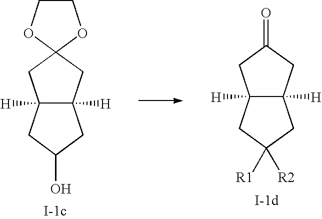
or reacting the obtained 3-hydroxy-7,7-(ethylidene acetal)bicyclo[3.3.0] octane (I-1c) with different isocyanate and trimethylchlorosilane at room temperature or with different Grignard reagent in the solvent of ether and then acidifying it further by 2N hydrochloric acid to give the compounds of the formula (I-1d); 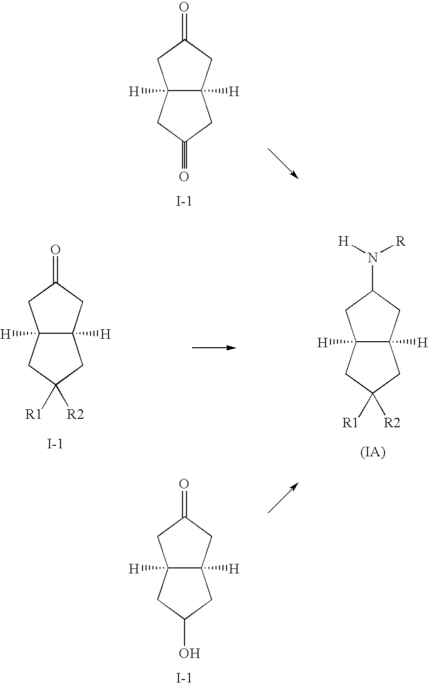
reacting the compounds of formula (I-1a) or formula (I-1d) or formula (I-1e) each independently with equivalent different amine in the solvent of methanol under the presence of sodium triethoxyborohydride and triethylamine at room temperature to give the compounds of formula (IA); wherein:
R is selected from the group consisting of alkyl, cycloalkyl, haloalkyl, aryl, heteroaryl, aminocarbonyl alkyl, amide alkyl, heterocyclo aminocarbonyl alkyl and aminoalkyl, wherein the heterocycle is selected from the group consisting of 5- or 6-membered hetero ring further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester and halogen;
R₁ and R₂ are each independently selected from the group consisting of hydrogen, hydroxyl, alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;
wherein R₁ and R₂ are attached together with the atom to form a 3 to 8 membered ring, wherein the 3 to 8 membered hetero ring further contains one or more heteroatoms selected from the group consisting of N, O and S, and the 3 to 8 membered hetero rings so formed are substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;
R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;
R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered hetero rings so formed are further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;
R₆ is selected from the group consisting of hydrogen or alkyl;
r is an integer from 1 to 6; and
m is an integer from 0 to 6.

9. The preparation process of claim 8, wherein:
R is heterocyclo aminocarbonyl alkyl, wherein the heterocycle is selected from the group consisting of 5- or 6-membered hetero ring further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester and halogen;
R₁ is hydrogen or hydroxyl, and R₂ is selected from the group consisting of hydrogen, hydroxyl, alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;
R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;
R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered hetero rings so formed are substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;
R₆ is selected from the group consisting of hydrogen or alkyl;
r is an integer from 1 to 6; and
m is an integer from 0 to 6.

10. The preparation process of claim 8, wherein R is the formula 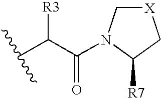
wherein, R₃ is selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups selected from the group consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, carboxylic acid and carboxylic ester;
R₇ is selected from the group consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heteroalkyl, carboxylic acid, carboxylic ester and halogen; and
X is C, S or O.

11. The preparation process of claim 8, wherein the preparation process also comprises that the obtained compounds of formula (IA) through purification are directly reacted with acids in the solvent of ether under an ice-water bath to give the acid addition salt thereof.

12. The preparation process of claim 8, wherein the preparation process also comprises that the obtained compounds of formula (IA) is reacted with di-tert-butyl dicarbonate to protect nitrogen atom, purified the compounds by silica gel column chromatography, then
reacted with acid in the solvent of ether under an ice-water bath to give the acid addition salt thereof.

13. The preparation process of claim 11 or 12, wherein the acids are hydrochloric acid, methanesulfonic acid, sulfuric acid, phosphoric acid, citric acid, acetic acid or trifluoroacetic acid.

14. The preparation process of claim 11 or 12, wherein the acids are hydrochloric acid or trifluoroacetic acid.

15. The preparation process of claim 8, wherein the compounds of formula (I-1a) or formula (I-1d) or formula (I-1e) is each independently reacted with equivalent RNH₂ in the solvent of methanol under the presence of sodium triethoxyborohydride and triethylamine at room temperature to give the compounds of formula (IA); wherein
R is selected from the group consisting of alkyl, cycloalkyl, haloalkyl, aryl, heteroaryl, aminocarbonyl alkyl, amide alkyl, heterocyclo aminocarbonyl alkyl and aminoalkyl, wherein the heterocycle is selected from the group consisting of 5- or 6-membered hetero ring further substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester and halogen;

16. The preparation process of claim 15, wherein R is the formula 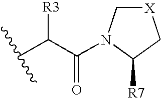
wherein, R₃ is selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups selected from the group consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, carboxylic acid and carboxylic ester; R₇ is selected from the group consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heteroalkyl, carboxylic acid, carboxylic ester and halogen; and
X is C, S or O.

17. The compounds or pharmaceutically acceptable salts thereof of claim 1, wherein the compounds are selected from the group consisting of:

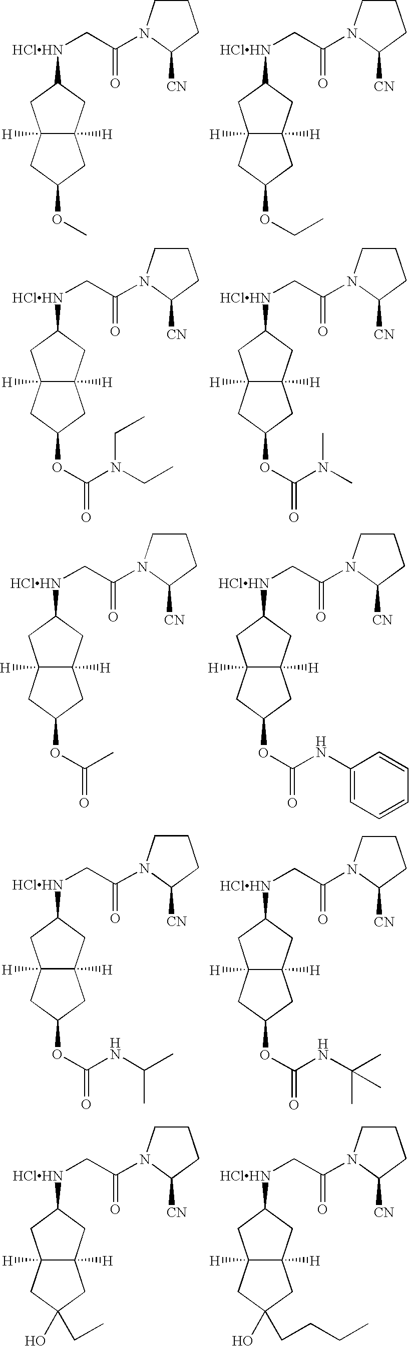

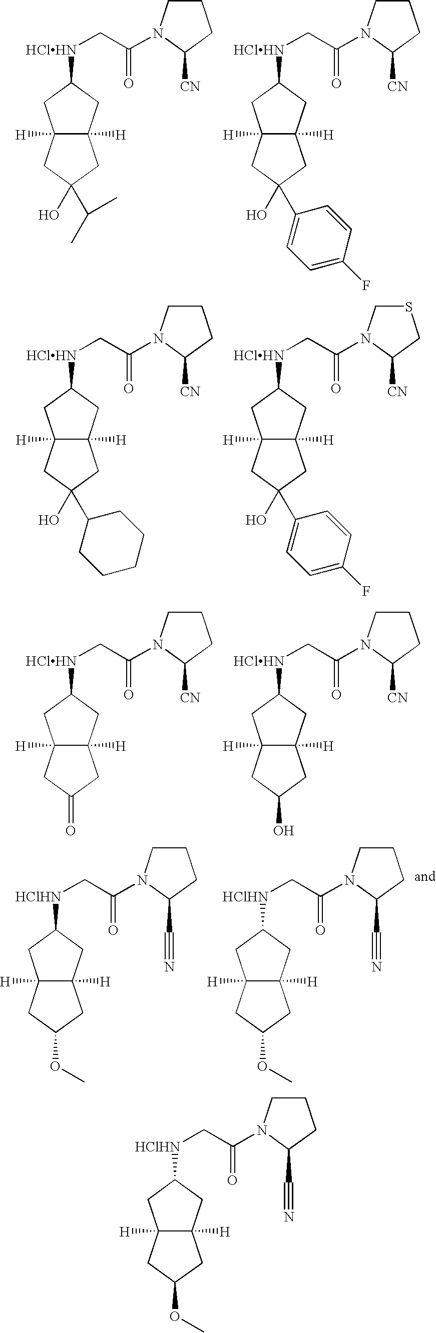

18. Compounds as intermediates in the synthesis of the compounds of claim 1, having the following formula (I-1d) or (I-1e): 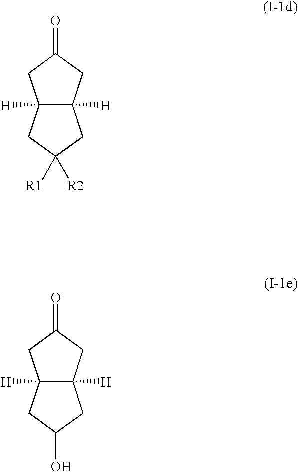
wherein:
R₁ and R₂ are each independently selected from the group consisting of alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ or —NR₄R₅, wherein the alkyl, cycloalkyl, heterocyclo alkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;
R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocycloalkoxyl, trifluoromethyl, carboxylic acid and carboxylic ester;
R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered hetero rings so formed are substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;
R₆ is selected from the group consisting of hydrogen or alkyl;
r is an integer from 1 to 6; and
m is an integer from 0 to 6.

19. A preparation process for the compounds of formula (I-1d) or (I-1e) of claim 18, comprising the following steps of: 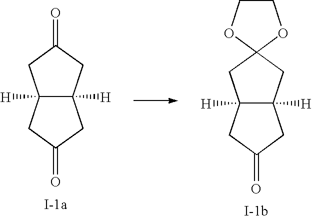
reacting starting material tetrahydro-pentalene-2,5-dione (I-1a) with ethylene glycol and a catalyst of p-toluenesulfonic acid through refluxing in the solvent of benzene to give 7,7-(ethylidene acetal)bicyclo[3.3.0] octane-3-one protected (I-1b); 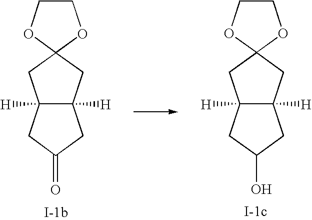
reducing the obtained 7,7-(ethylidene acetal)bicyclo[3.3.0] octane-3-one (I-1b) by NaBH₄ at room temperature to afford 3-hydroxy-7,7-(ethylidene acetal)bicyclo[3.3.0] octane (I-1c); 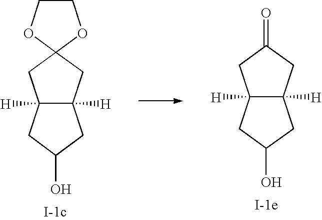
reacting the obtained 3-hydroxy-7,7-(ethylidene acetal)bicyclo[3.3.0] octane (I-1c) with oxalic acid in the mixed solvent of ethyl acetate and water to give 5-hydroxy-hexahydro-pentalen-2-one (I-1e); 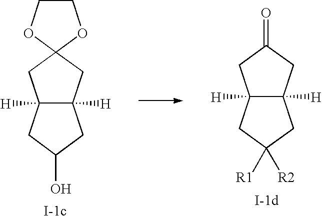
or reacting the obtained 3-hydroxy-7,7-(ethylidene acetal)bicyclo[3.3.0] octane (I-1c) with different isocyanate and trimethylchlorosilane at room temperature or with different Grignard reagent in the solvent of ether and then acidifying it further by 2N hydrochloric acid to give the compounds of the formula (I-1d);
wherein:
R₁ and R₂ are each independently selected from the group consisting of alkyl, cycloalkyl, heterocyclo alkyl, aryl, heteroaryl, —OR₄, —(CH₂CH₂O)ᵣR₆, —(CH₂)ₘC(O)OR₄, —(CH₂)ₘC(O)NR₄R₅, —(CH₂)ₘOC(O)NR₄R₅, —C(O)R₄, —NR₆C(O)R₅, —NR₄C(O)OR₅, —OC(O)OR₄, —OC(O)NR₄R₅, —NC(O)NR₄R₅ and —NR₄R₅, wherein the alkyl, cycloalkyl, heterocyclo alkyl, aryl or heteroaryl is further substituted by one or more groups consisting of alkyl, halogen, aryl, hydroxyl, amino, alkyl amino, amide group, alkoxyl, aryloxyl, heterocyclo alkyl, carboxylic acid and carboxylic ester;
R₄ and R₅ are each independently selected from the group consisting of hydrogen, alkyl, cycloalkyl, aryl, heteroaryl and heterocyclo alkyl, wherein the alkyl, cycloalkyl, aryl, heteroaryl or heterocyclo alkyl is further substituted by one or more groups consisting of alkyl, cycloalkyl, aryl, heteroaryl, alkoxyl, cycloalkoxyl, aryloxyl, heteroaryloxyl, halogen, hydroxyl, amino, alkylamino, amide group, aminocarbonyl, cyano, aminoalkyl, hydroxyalkyl, heterocyclo alkyl, heterocyclo alkoxyl, trifluoromethyl, carboxylic acid and carboxylic ester;
R₄ and R₅ are attached together with the N atom to form the 3 to 8 membered hetero ring, wherein the 5 to 8 membered hetero ring further contains one or more heteroatoms selected from the group consisting of N, O and S, and the 3 to 8 membered hetero rings so formed are substituted by one or more groups consisting of alkyl, aryl, heteroaryl, haloalkyl, haloalkoxyl, hydroxyl, amino, alkylamino, amide group, aminocarbonyl, cyano, alkoxyl, aryloxyl, aminoalkyl, hydroxyalkyl, heterocyclo alkyl, carboxylic acid, carboxylic ester, halogen and —NR₄R₅;
R₆ is selected from the group consisting of hydrogen or alkyl;
r is an integer from 1 to 6;
m is an integer from 0 to 6.

20. A pharmaceutical composition comprising a compound of any one of claims 1-7 and 17 or salt thereof in an effective therapeutic dose and a pharmaceutically acceptable carrier.

21. A dipeptidyl peptidase IV (DPPIV) inhibitor pharmaceutical composition comprising a compound of claim 1 or salt thereof.

22. A dipeptidyl peptidase IV (DPPIV) inhibitor pharmaceutical composition comprising an effective amount of a compound of any one of claims 1-7 and 17 or salt thereof and a pharmaceutically acceptable carrier.
